SEQUENCE LISTING (19 sequences)

SEQ ID NO: 1 - DNA, 18 bp, Artificial Sequence
  NOBEL phosphorothioate AS ODN
  misc_feature (1)..(18): May be joined by a phosphorothioate linkage
tcctccggagccagactt

SEQ ID NO: 2 - DNA, 17 bp, Artificial Sequence
  Recombinant Insulin-like Growth Factor Receptor (IGF-1R) antisense oligodeoxynucleotide (AS-ODN)
ttctccactcgtcggcc

SEQ ID NO: 3 - DNA, 18 bp, Artificial Sequence
  Recombinant Insulin-like Growth Factor Receptor (IGF-1R) antisense oligodeoxynucleotide (AS-ODN)
acaggccgtgtcgttgtc

SEQ ID NO: 4 - DNA, 18 bp, Artificial Sequence
  Recombinant Insulin-like Growth Factor Receptor (IGF-1R) antisense oligodeoxynucleotide (AS-ODN)
gcactcgccgtcgtggat

SEQ ID NO: 5 - DNA, 18 bp, Artificial Sequence
  Recombinant Insulin-like Growth Factor Receptor (IGF-1R) antisense oligodeoxynucleotide (AS-ODN)
cggatatggtcgttctcc

SEQ ID NO: 6 - DNA, 18 bp, Artificial Sequence
  Recombinant Insulin-like Growth Factor Receptor (IGF-1R) antisense oligodeoxynucleotide (AS-ODN)
tctcagcctcgtggttgc

SEQ ID NO: 7 - DNA, 18 bp, Artificial Sequence
  Recombinant Insulin-like Growth Factor Receptor (IGF-1R) antisense oligodeoxynucleotide (AS-ODN)
ttgcggcctcgttcactg

SEQ ID NO: 8 - DNA, 18 bp, Artificial Sequence
  Recombinant Insulin-like Growth Factor Receptor (IGF-1R) antisense oligodeoxynucleotide (AS-ODN)
aagcttcgttgagaaact

SEQ ID NO: 9 - DNA, 18 bp, Artificial Sequence
  Recombinant Insulin-like Growth Factor Receptor (IGF-1R) antisense oligodeoxynucleotide (AS-ODN)
ggacttgctcgttggaca

SEQ ID NO: 10 - DNA, 18 bp, Artificial Sequence
  Recombinant Insulin-like Growth Factor Receptor (IGF-1R) antisense oligodeoxynucleotide (AS-ODN)
ggctgtctctcgtcgaag

SEQ ID NO: 11 - DNA, 20 bp, Artificial Sequence
  IDT1220 phosphorothioate AS ODN
  misc_feature (1)..(20): May be joined by a phosphorothioate linkage
cagatttctccactcgtcgg

SEQ ID NO: 12 - DNA, 17 bp, Artificial Sequence
  Recombinant Insulin-like Growth Factor Receptor (IGF-1R) antisense oligodeoxynucleotide (AS-ODN)
ccggagccagacttcat

SEQ ID NO: 13 - DNA, 20 bp, Artificial Sequence
  Recombinant Insulin-like Growth Factor Receptor (IGF-1R) antisense oligodeoxynucleotide (AS-ODN)
ctgctcctcctctaggatga

SEQ ID NO: 14 - DNA, 15 bp, Artificial Sequence
  Recombinant Insulin-like Growth Factor Receptor (IGF-1R) antisense oligodeoxynucleotide (AS-ODN)
ccctcctccggagcc

SEQ ID NO: 15 - DNA, 18 bp, Artificial Sequence
  DWA phosphorothioate AS ODN
  misc_feature (1)..(18): May be joined by a phosphorothioate linkage
ggaccctcctccggagcc

SEQ ID NO: 16 - DNA, 18 bp, Artificial Sequence
  DWA locked nucleic acid AS ODN
  misc_feature (1)..(6): May be joined by a locked nucleic acid linkage
  misc_feature (6)..(13): May be joined by a phosphodiester linkage
  misc_feature (13)..(18): May be joined by a locked nucleic acid linkage
ggaccctcctccggagcc

SEQ ID NO: 17 - DNA, 18 bp, Artificial Sequence
  DWA phosphodiester AS ODN
  misc_feature (1)..(18): May be joined by a phosphodiester linkage
ggaccctcctccggagcc

SEQ ID NO: 18 - RNA, 927 nt, Homo sapiens
cuuuguuuucuuuucuuccucacagaccuucgggcaaggaccuucacaagggaugcaguacaugcucuggcugccguugcggaugaagcccgaggggcacuccugcaugcacucgccgucguggaucacaaaccccucggagucgcugcucucggcgcugaggauguuggcgcagaagucacgguccacacagcgccagcccucaaaccuguagguguugggcgggcaggcaggcacacagacaccggcauaguaguaguggcggcaagcuacacaggccgugucguugucaggcgcgcugcagcugcccaggcacucgggguggcagcacucauuguucucggugcacgcccgcuucccacacgugcuugggcacauuuucuggcagcgguuugugguccagcagcgguaguuguacucauuguugaugguggucuucucacacaucggcuucuccuccauggucccuggacacagguccccacauuccuuugggggcuuauuccccacaauguaguuauuggacaccgcauccaggaucagggaccaguccacaguggagagguaacagaggucagcauuuuucacaauccugauggccccccgaguaauguuccucagguuguaaagcccaauauccuugagauuggucaucucgaagaugaccagggcguaguuguagaagaguuuccagccgcggaugaccgugagguuggggaagaggucuccgaggcucucgaggccagccacucggaacagcagcaaguacucgguaaugaccgugagcuuggggaagcgguagcugcgguaguccucggccuuggagaugagcaggauguggagguagcccucgaucaccgugcaguucuccaggcgcuucagcugcugauagucguugcggaugucgaugccuggcccgcagauuuc

SEQ ID NO: 19 - DNA, 4104 bp, Homo sapiens
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

(19) United States
(12) Patent Application Publication
(10) Pub. No.: US 2022/0195440 A1
(43) Pub. Date: Jun. 23, 2022

(54) METHODS FOR TREATING CANCERS USING ANTISENSE

(71) Applicant: Thomas Jefferson University, Philadelphia, PA (US)

(72) Inventors: David W. Andrews, Philadelphia, PA (US); Douglas C. Hooper, Medford, NJ (US)

(21) Appl. No.: 17/599,023

(22) PCT Filed: Mar. 27, 2020
(86) PCT No.: PCT/US20/25217

§ 371 Date: Sep. 28, 2021

Related U.S. Application Data
(60) Provisional application No. 62/825,516, filed on Mar. 28, 2019.

(51) Int. Cl. C12N 15/113; G01N 33/574; A61K 31/495

(52) U.S. Cl. CPC C12N 15/1138; G01N 33/57484; C12N 2310/11; C12N 2320/31; A61K 31/495

(57) ABSTRACT

The present disclosure relates to compositions and methods for treating cancers using antisense (AS) nucleic acids directed against Insulin-like Growth Factor 1 Receptor (IGF-1R). The AS may be administered to the patients systemically, or may be used to produce an autologous cancer cell vaccine. In some embodiments, the AS are provides in an implantable irradiated biodiffusion chamber comprising tumor cells and an effective amount of the AS. The chambers are irradiated and implanted in the abdomem of subjects and stimulate an immune response that attacks tumors distally. The compositions and methods disclosed herein may be used to treat many different kinds of cancer, for example glioblastoma. In some embodiments, the method are provided to predict the effectiveness of antisense (AS) nucleic acids directed against Insulin-like Growth Factor 1 Receptor (IGF-1R) in a subject.

The present disclosure relates to compositions and methods for treating cancers using antisense nucleic acids directed against Insulin-like Growth Factor-1 Receptor (IGF-1R). The present disclosure also relates to compositions and methods for treating cancers by treating subjects with at least one implantable irradiated biodiffusion chamber (see U.S. Pat. No. 6,541,036 and PCT/US2016/026970, which are incorporated herein by reference in their entireties) comprising tumor cells and an antisense nucleic acid directed against IGF-1R.

### FIELD OF THE INVENTION

### Description of the Text File Submitted Electronically

The contents of the text file submitted electronically herewith are incorporated by reference in their entirety: a computer readable format copy of the Sequence Listing (filename: IMVX-014-00US_SEQUENCE_LISTING.txt, date recorded Mar. 28, 2019, file size ˜12 kilobytes).

### Background

PCT Patent Application Publication Number WO/2018/165528 (hereby incorporated by reference in its entirety) discloses “compositions and methods for treating cancers using antisense (AS) nucleic acids directed against Insulin-like Growth Factor 1 Receptor (IGF-1R).” The AS may be administered to the patients systemically, or may be used to produce an autologous cancer cell vaccine. Because patient responses to the IGF-1R may vary, there is a need for methods of predicting the prognosis of a subject having cancer in response to treatment with an IGF-1R AS ODN.

### SUMMARY

The present disclosure relates at least in part to the use of an antisense oligodeoxynucleotide (AS-ODN) targeting the insulin-like growth factor receptor-1 (IGF-1R) (“IGF-1R AS ODN”) to treat a subject having cancer. In particular aspects, the disclosure relates methods and compositions (including diagnostics and companion diagnostics) pertaining to identification of subjects that have an increased likelihood of responding to treatment with an IGF-1R AS ODN and administering an IGF-1R AS ODN to such subjects. In certain embodiments, subjects that have an increased likelihood of responding to treatment with an IGF-1R AS ODN are identified by determining MGMT methylation and/or determining the T-cell function in said subject. In certain embodiments, subjects that have an increased likelihood of responding to treatment with an IGF-1R AS ODN are identified by determining MGMT methylation and/or determining the T-cell function in said subject; wherein patients having methylated MGMT and/or having good T-cell function in the subject is indicative of an increased likelihood of responding to treatment with an IGF-1R AS ODN.

In some embodiments, provided is a method of predicting the prognosis of a subject having cancer (e.g., glioma or glioblastoma) in response to treatment with an IGF-1R AS ODN; wherein the method involves determining the MGMT methylation and/or determining the T-cell function in said subject.

In one aspect provided is a method that includes determining the MGMT methylation and/or determining the T-cell function in a subject diagnosed with cancer and subsequently administering an IGF-1R AS ODN to the subject. In a related embodiment provided is a method includes determining the MGMT methylation and/or determining the T-cell function in a subject diagnosed with cancer and subsequently administering a an IGF-1R AS ODN to the subject only if the subject is identified as having methylated MGMT and/or if the subject has good T-cell function.

As used herein MGMT refers to 06-methylguanine-DNA methyltransferase (e.g., Uniprot Accession No. Q6LDD1). Methylation of the 06-methylguanine-DNA methyltransferase (MGMT) promoter silences the ability of a cell to dealkylate the methyl group on 06 guanine and increases the therapeutic efficacy of temozolomide (TMZ) compared to patients with an unmethylated MGMT promoter. DNA methylation, which is the covalent addition of a methyl group usually at the 5′-position of a cytosine or guanine nucleotide. Evaluation of MGMT methylation can be performed and determined using methods well known in the art. In some embodiments the methylation status of eight CpG islands within the MGMT gene promoter is evaluated. Methylated MGMT in various embodiments is indicative of a favorable prognosis and or/expectation of a positive response to IGF-1R AS ODN treatment; whereas unmethylated MGMT in various embodiments is indicative of a favorable prognosis and or/expectation of a positive response to IGF-1R AS ODN treatment.

Determination of T cell function can also be determined using methods and criteria as is well known in the art. In some embodiments T cell function is determined by evaluating the number of T cells expressing IFN-γ in response to nonspecific stimulation. In certain embodiments, the term “good T cell function” as used herein refers to subjects with a median or greater number of T cells expressing IFN-γ in response to nonspecific stimulation; and the term “poor T cell function” refers to a subject with less than median or lessor number of T cells expressing IFN-γ in response to nonspecific stimulation.

In some embodiments, said IGF-1R AS ODN is administered to subject before temozolamide is administered to said subject. In certain embodiments, said IGF-1R AS ODN is administered to said subject at least 2 weeks; at least 3 weeks; least 4 weeks; at least 5 weeks; at least 6 weeks; at least 7 weeks; or at least 8 weeks before temozolomide is administered to said subject.

In some embodiments of any of the aspects and embodiments as provided herein the IGF-1R AS ODN is administered to the subject as an autologous cancer cell vaccine. In some embodiments of any of the aspects and embodiments as provided herein the IGF-1R AS ODN is administered to the subject as a fully formulated biodiffusion chamber.

The present disclosure demonstrates that an antisense oligodeoxynucleotide (AS-ODN) targeting the insulin-like growth factor receptor-1 (IGF-1R) effectively stimulates a response in a subject that treats cancer when used in the therapeutic approaches described herein. In particular aspects, methods are effective for treating cancer in a patient as part of an autologous cancer cell vaccine alone or, optionally, along with systemic administration. In preferred approaches, the methods disclosed herein provide effective cancer therapy as a monotherapy; i.e. in the absence of chemotherapy and in the absence of radiation therapy.

In embodiments, the present disclosure provides a biodiffusion chamber for implantation into a subject suffering from a tumor, the biodiffusion chamber comprising irradiated tumor cells and irradiated insulin-like growth factor receptor-1 antisense oligodeoxynucleotide (IGF-1R AS ODN). In embodiments, the tumor cells are removed from a resection site of the subject.

In embodiments, the present disclosure provides a diffusion chamber comprising irradiated IGF-1R AS ODN and irradiated, adhesion-enriched, morselized tumor cells; wherein the biodiffusion chamber comprises a membrane that is impermeable to the cells and permeable to the IGF-1R AS ODN.

In embodiments, the tumor cells are removed from the resection site using an endoscopic device. In further embodiments, the tumor cells are removed from the resection site using a tissue morselator. In some embodiments, the tumor cells are viable when removed from the resection site using the tissue morselator. In other embodiments, the tissue morselator comprises a high-speed reciprocating inner cannula within a stationary outer cannula. The outer cannula may comprise a side aperture, and further wherein the tumor cells are drawn into the side aperture by electronically controlled variable suction. In embodiments, the tissue morselator does not produce heat at the resection site. In still further embodiments, the tumor cells are enriched for nestin expression before they are placed into the biodiffusion chamber. In some embodiments, implantation of the chamber inhibits regrowth of the tumor in the subject. In some embodiments, implantation of the chamber inhibits regrowth of the tumor for at least 3 months, at least 6 months, at least 12 months, or at least 36 months.

In additional embodiments, the present disclosure provides a method for preparing a biodiffusion chamber for implantation into a subject suffering from a tumor, the method comprising placing tumor cells into the biodiffusion chamber in the presence of an IGF-1R AS ODN, and irradiating the biodiffusion chamber, wherein the tumor cells are removed from a resection site in the subject using a tissue morselator that does not produce heat at the resection site. Typically, multiple chambers are used. For example, about 10 chambers, or about 20 chambers. Advantageously, an optimal anti-tumor response is obtained when the number of cells in the chamber is about 750.000 to about 1,250,000; for example about 1,000,000 per chamber where 20 chambers are implanted.

In some embodiments, the tissue morselator is an endoscopic device. In further embodiments, the tissue morselator comprises a high-speed reciprocating inner cannula within a stationary outer cannula. In additional embodiments, the outer cannula comprises a side aperture, and the tumor cells are drawn into the side aperture by electronically controlled variable suction.

In embodiments, the present disclosure provides a method of treating a subject suffering from a tumor, the method comprising implanting one or more biodiffusion chambers into the subject, wherein the one or more biodiffusion chambers comprise irradiated tumor cells, and irradiated insulin-like growth factor receptor-1 antisense oligodeoxynucleotide (IGF-1R AS ODN), wherein the tumor cells are removed from a resection site in the subject using a tissue morselator that does not produce heat at the resection site.

### DETAILED DESCRIPTION

### Definitions

All terms not defined herein have their common art-recognized meanings.

As used herein, terms such as “a,” “an,” and “the” include singular and plural referents unless the context clearly demands otherwise.

As used herein, the term “about” when preceding a numerical value indicates the value plus or minus a range of 10%. For example, “about 100” encompasses 90 and 110. For the avoidance of doubt, it is understood that the term about includes the indicated value itself in addition to the 10% range, for example “about 100” includes exactly 100 as well as the range of 90-100.

As used herein, the term “autologous” means cells or tissues obtained from the same individual.

As used herein, the term “autologous cancer cell vaccine” refers to a therapeutic produced in part by isolating tumor cells from an individual and processing these tumor cells ex vivo. The cells are then re-administered to the individual from whom the tumor cells were isolated. In embodiments, an autologous cancer cell vaccine may comprise additional components in addition to the tumor cells, such as a buffer and/or antisense nucleic acids (such as, for example IGF-1R AS ODN). In embodiments, “autologous cancer cell vaccine” may refer to a biodiffusion chamber containing the tumor cells and one or more additional components. In certain aspects, the “autologous cancer cell vaccine” may be a “fully formulated chamber” also referred to herein as “fully formulated biodiffusion chamber.”

As used herein, the term “fully formulated chamber” or “fully formulated biodiffusion chamber” is a biodiffusion chamber that includes autologous tumor cells and other cells included in the tumor microenvironment (TME) that may or may not be treated prior to encapsulation in the chamber with a first amount of an IGF-1R AS ODN. The cells are encapsulated with exogenous addition of a second amount, for example at least 2 μg, of IGF-1R AS ODN and the chamber is then irradiated with 5 Gy of gamma-irradiation.

As used herein, the term “small molecules” includes nucleic acids, peptides, proteins, and other chemicals (such as, for example, cytokines and growth hormones produced by cells), but does not include cells, exosomes, or microvesicles.

The term “targeting IGF-1R expression” or “targets IGF-1R expression” as used herein refers to administering an antisense nucleic acid that has a sequence designed to bind to the IGF-1R.

As used herein, the term “systemic administration” refers to achieving delivery of a substance throughout the body of a subject. Typical systemic routes of administration include parenteral administration, transdermal administration, intraperitoneal administration, intravenous administration, subcutaneous administration, and intramuscular administration.

Other administration routes include oral administration, nasal administration topical administration, intraocular administration, buccal administration, sublingual administration, vaginal administration, intraheptic, intracardiac, intrapancreatic, by inhalation, and via an implanted pump.

Antisense Molecules

Antisense molecules are nucleic acids that work by binding to a targeted complimentary sequence of mRNA by Watson and Crick base-pairing rules. The translation of target mRNA is inhibited by an active and/or passive mechanism when hybridization occurs between the complementary helices. In the passive mechanism, hybridization between the mRNA and exogenous nucleotide sequence leads to duplex formation that prevents the ribosomal complex from reading the message. In the active mechanism, hybridization promotes the binding of RnaseH, which destroys the RNA but leaves the antisense intact to hybridize with another complementary mRNA target. Either or both mechanisms inhibit translation of a protein contributing to or sustaining a malignant phenotype. As therapeutic agents, antisense molecules are far more selective and as a result, more effective and less toxic than conventional drugs.

The methods and compositions disclosed herein involve the use of antisense molecules for treating cancer. Typically, the antisense molecule is an antisense oligodeoxynucleotide (AS-ODN). In some embodiments, the antisense molecule comprises a modified phosphate backbone. In certain aspects, the phosphate backbone modification renders the antisense more resistant to nuclease degradation. In certain embodiments, the modification is a locked antisense. In other embodiments, the modification is a phosphorothioate linkage. In certain aspects, the antisense contains one or more phosphorothioate linkages. In certain embodiments, the phosphorothioate linkages stabilize the antisense molecule by conferring nuclease resistance, thereby increasing its half-life. In some embodiments, the antisense may be partially phosphorothioate-linked. For example, up to about 1%, up to about 3%, up to about 5%, up to about 10%, up to about 20%, up to about 30%, up to about 40%, up to about 50% up to about 60%, up to about 70%, up to about 80%, up to about 90%, up to about 95%, or up to about 99% of the antisense may be phosphorothioate-linked. In some embodiments, the antisense is fully phosphorothioate-linked. In other embodiments, phosphorothioate linkages may alternate with phosphodiester linkages. In certain embodiments, the antisense has at least one terminal phosphorothioate monophosphate.

In some embodiments, the antisense molecule comprises one or more CpG motifs. In other embodiments, the antisense molecule does not comprise a CpG motif. In certain aspects, the one or more CpG motifs are methylated. In other aspects, the one or more CpG motifs are unmethylated. In certain embodiments, the one or more unmethylated CpG motifs elicit an innate immune response when the antisense molecule is administered to a subject. In some aspects, the innate immune response is mediated by binding of the unmethylated CpG-containing antisense molecule to Toll like Receptors (TLR).

In certain embodiments, the antisense molecule comprises at least one terminal modification or “cap”. The cap may be a 5′ and/or a 3′-cap structure. The terms “cap” or “end-cap” include chemical modifications at either terminus of the oligonucleotide (with respect to terminal ribonucleotides), and including modifications at the linkage between the last two nucleotides on the 5′ end and the last two nucleotides on the 3′ end. The cap structure may increase resistance of the antisense molecule to exonucleases without compromising molecular interactions with the target sequence or cellular machinery. Such modifications may be selected on the basis of their increased potency in vitro or in vivo. The cap can be present at the 5% terminus (5′-cap) or at the 3′-terminus (3′-cap) or can be present on both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety. The phosphorothioate or phosphorodithioate linkage(s), when part of a cap structure, are generally positioned between the two terminal nucleotides on the 5′ end and the two terminal nucleotides on the 3′ end.

In preferred embodiments, the antisense molecule targets the expression of Insulin like Growth Factor 1 Receptor (IGF-1R). IGF-1R is a tyrosine kinase cell surface receptor that shares 70% homology with the insulin receptor. When activated by its ligands (IGF-I, IGF-II and insulin), it regulates broad cellular functions including proliferation, transformation and cell survival. The IGF-1R is not an absolute requirement for normal growth, but it is essential for growth in anchorage-independent conditions that may occur in malignant tissues. A review of the role of IGF-1R in tumors is provided in Baserga et al., Vitamins and Hormones, 53:65-98 (1997), which is incorporated herein by reference in its entirety.

In certain embodiments, the antisense molecule is an oligonucleotide directed against DNA or RNA of a growth factor or growth factor receptor, such as, for example, IGF-1R.

In certain embodiments, the antisense is a deoxynucleotide directed against IGF-1R (IGF-1R AS ODN). The full length coding sequence of IGF-1R is provided as SEQ ID NO:19 (see, for example, PCUUS2016/26970, which is incorporated herein by reference in its entirety).

In certain embodiments, the antisense molecule comprises nucleotide sequences complementary to the IGF-1R signal sequence, comprising either RNA or DNA. The signal sequence of IGF-1R is a 30 amino acid sequence. In other embodiments, the antisense molecule comprises nucleotide sequences complementary to portions of the IGF-1R signal sequence, comprising either RNA or DNA. In some embodiments, the antisense molecule comprises nucleotide sequences complementary to codons 1-309 of IGF-1R, comprising either RNA or DNA. In other embodiments, the antisense molecule comprises nucleotide sequences complementary to portions of codons 1-309 of IGF-1R, comprising either RNA or DNA.

In certain embodiments, the IGF-1R AS ODN is at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, or at least about 50 nucleotides in length. In some embodiments, the IGF-1R AS ODN is from about 15 nucleotides to about 22 nucleotides in length. In certain aspects, the IGF-1R AS ODN is about 18 nucleotides in length.

In certain embodiments, the IGF-1R AS ODN forms a secondary structure at 18° C., but does not form a secondary structure at about 37° C. In other embodiments, the IGF-1R AS ODN does not form a secondary structure at about 18° C. or at about 37° C. In yet other embodiments, the IGF-1R AS ODN does not form a secondary structure at any temperature. In other embodiments, the IGF-1R AS ODN does not form a secondary structure at 37° C. In particular embodiments, the secondary structure is a hairpin loop structure.

In some aspects, the IGF-1R AS ODN comprises the nucleotide sequence of SEQ ID NO:1, or a fragment thereof. In certain embodiments, the IGF-1R AS ODN may have at least about 70%, at least about 75%, at least about 80%, at least about 85%, at least about 90%, at least about 95%, at least about 96%, at least about 98%, or 100% identity to SEQ ID NO: 1, or a fragment thereof. In some embodiments, the IGF-1R AS ODN comprises one or more phosphorothioate linkages.

In certain aspects, the IGF-1R AS ODN consists of SEQ ID NO: 1. NOBEL is an 18-mer oligodeoxynucleotide with a phosphorothioate backbone and a sequence complimentary to codons 2 through 7 in the IGF-1R gene. As such, NOBEL is an antisense oligonucleotide directed against IGF-1R (IGF-1R AS ODN). The NOBEL sequence, derived as the complimentary sequence of the IGF-1R gene at the 5′ end, is:

(SEQ ID NO: 1)
5′-TCCTCCGGAGCCAGACTT-3′.

NOBEL has a stable shelf life and is resistant to nuclease degradation due to its phosphorothioate backbone. Administration of NOBEL can be provided in any of the standard methods associated with introduction of oligodeoxynucleotides known to one of ordinary skill in the art. Advantageously, the AS ODNs disclosed herein, including NOBEL, may be administered with little/no toxicity. Even levels of about 2 g/kg (scaled) based on mice tests (40 μg in the tail vain) did not reveal toxicity issues. NOBEL can be manufactured according to ordinary procedures known to one of ordinary skill in the art.

The antisense molecule, for example the NOBEL sequence of SEQ ID NO: 1, may also comprise one or more p-ethoxy backbone modifications as disclosed in U.S. Pat. No. 9,744,187, which is incorporated by reference herein in its entirety. In some embodiments, the nucleic acid backbone of the antisense molecule comprises at least one p-ethoxy backbone linkage. For example, up to about 1%, up to about 3%, up to about 5%, up to about 10%, up to about 20%, up to about 30%, up to about 40%, up to about 50% up to about 60%, up to about 70%, up to about 80%, up to about 90%, up to about 95%, or up to about 99% of the antisense molecule may be p-ethoxy-linked. The remainder of the linkages may be phosphodiester linkages or phosphorothioate linkages or a combination thereof. In a preferred embodiment 50% to 80% of the phosphate backbone linkages in each oligonucleotide are p-ethoxy backbone linkages, wherein 20% to 50% of the phosphate backbone linkages in each oligonucleotide are phosphodiester backbone linkages.

Various IGF-1R antisense sequences are bioactive in some or all of the multi-modality effects of the NOBEL sequence. The 18-mer NOBEL sequence has both IGF-1R receptor downregulation activity as well as TLR agonist activity, and further experimentation in mice suggests that both activities are necessary for in vivo anti-tumor immune activity. While the AS ODN molecule has anti-tumor activity, the complimentary sense sequence does not, despite also having a CpG motif.

In certain embodiments, the sequence of the antisense is selected from the group consisting of SEQ ID NOS 1-14, as shown in Table 1. In some embodiments, the antisense has 90% sequence identity to one or more of SEQ ID NOS 1-14. In some embodiments, the antisense has 80% sequence identity to one or more of SEQ ID NOS 1-14. In some embodiments, the antisense has 70% sequence identity to one or more of SEQ ID NOS 1-14.

TABLE 1
Additional downstream sequences
for IGF-1R AS ODN Formulation

| | Corresponds to | SEQ |
|---|---|---|
| Sequences with ACGA Motif | IGF-1R Codons | ID NO: |
| 5′-TCCTCCGGAGCCAGACTT-3′ | 2-7 | 1 |
| 5′-TTCTCCACTCGTCGGCC-3′ | 26-32 | 2 |
| 5′-ACAGGCCGTGTCGTTGTC-3′ | 242-248 | 3 |
| 5′-GCACTCGCCGTCGTGGAT-3′ | 297-303 | 4 |
| 5′-CGGATATGGTCGTTCTCC-3′ | 589-595 | 5 |
| 5′-TCTCAGCCTCGTGGTTGC-3′ | 806-812 | 6 |
| 5′-TTGCGGCCTCGTTCACTG-3′ | 1,033-1,039 | 7 |
| 5′-AAGCTTCGTTGAGAAACT-3′ | 1,042-1,048 | 8 |
| 5′-GGACTTGCTCGTTGGACA-3′ | 1,215-1,221 | 9 |
| 5′-GGCTGTCTCTCGTCGAAG-3′ | 1,339-1,345 | 10 |
| 5′-CAGATTTCTCCACTCGTCGG-3′ | 27-34 | 11 |
| 5′-CCGGAGCCAGACTTCAT-3′ | 1-6 | 12 |
| 5′-CTGCTCCTCCTCTAGGATGA-3′ | 407-413 | 13 |
| 5′-CCCTCCTCCGGAGCC-3′ | 4-8 | 14 |

In certain embodiments, the IGF-1R AS ODN comprises the nucleotide sequence of any one of SEQ ID NOs:1-14, or fragments thereof. In certain embodiments, the IGF-1R AS ODN may have at least about 70%, at least about 75%, at least about 80%, at least about 85%, at least about 90%, at least about 95%, at least about 96%, at least about 98%, or 100% identity to any one of SEQ ID NOs: 1-14, or fragments thereof.

In some embodiments, the antisense molecule downregulates the expression of genes downstream of IGF-1R pathway in a cell. In certain aspects, the downstream gene is hexokinase (Hex II). In some embodiments, the antisense molecule downregulates the expression of housekeeping genes in the cell. In some aspects, the housekeeping gene is L13.

In certain aspects, the IGF-1R AS ODN is chemically synthesized. In certain embodiments, the IGF-1R AS ODN is manufactured by solid phase organic synthesis. In some aspects, the synthesis of the IGF-1R AS ODN is carried out in a synthesizer equipped with a closed chemical column reactor using flow-through technology. In some embodiments, each synthesis cycle sequence on the solid support consists of multiple steps, which are carried out sequentially until the full-length IGF-1R AS ODN is obtained. In certain embodiments, the IGF-1R AS ODN is stored in a liquid form. In other embodiments, the IGF-1R AS ODN is lyophilized prior to storing. In some embodiments, the lyophilized IGF-1R AS ODN is dissolved in water prior to use. In other embodiments, the lyophilized IGF-1R AS ODN is dissolved in an organic solvent prior to use. In yet other embodiment, the lyophilized IGF-1R AS ODN is formulated into a pharmaceutical composition. In some aspects the pharmaceutical composition is a liquid pharmaceutical composition. In other aspects, the pharmaceutical composition is a solid pharmaceutical composition. Additional antisense nucleic acids are also described in U.S. Publication No. 2017/0056430, which is incorporated herein by reference in its entirety.

Autologous Cancer Cell Vaccine

Introduction

Immunotherapy is currently used to target hematologic malignancies with one common cellular antigen. Unfortunately, solid tumors are far more complex, representing epigenetic progression of genetic changes to a malignant state with an unidentifiable number of tumor-specific targets. Even more challenging, within a WHO diagnostic cancer group there exists marked variations in tumor phenotypes. An autologous cell vaccine would encompass all such variations and all such targets and represent an ideal subject-specific immunotherapy for solid tumor cancers. An autologous cancer cell vaccine however, cannot be derived from primary cell cultures because serial passages alter the tumor phenotype thus diminishing the array of tumor-specific antigens. This would also require impossible lot-release qualification at each passage. The present disclosure eliminates these concerns by plating freshly resected, morselized tumor cells and reimplanting them within 24 hours as a depot antigen. In certain aspects, the excellent results achieved herein are obtained by ensuring that an appropriate number of cells are present in the chamber(s), among other specifics described herein.

Previous studies have designed autologous cell vaccine through the use of antigen presenting cells, instead of autologous tumor cells. In this paradigm, a subject's monocytes are collected from a pre-treatment plasma leukapheresis and differentiated into autologous dendritic cells (DC) ex vivo. The dendritic cells are then presented with the subject's tumor crude lysate inducing DC activation/maturation, and at a later time point, the matured dendritic cells, now cross-primed with tumor antigens are injected in the subject as a DC vaccine. Ex vivo differentiation, however, is missing a number of key stimulatory components only occurring in vivo. In addition, differentiation of DCs from hematopoietic precursors requires extensive in vitro manipulations with labor-intensive cell processing in expensive facilities. The present disclosure obviates these concerns by providing an endogenous DC maturation process and an immunomodulatory and immunostimulatory antisense oligodeoxynucleotide (AS-ODN) that promotes the development of an appropriate immune response. More specifically, the present disclosure provides a biodiffusion chamber comprising dispersed tumor cells derived from the patient and irradiated antisense molecules, which is implanted into the patient for therapeutically effective time. Without being bound by any theory, it is thought that the combination of irradiated tumor cells, antisense, and biodiffusion chamber act in concert to simulate the local immune response, and enhance the response by reducing or eliminating M2 cells, preventing dampening of the immune system.

Thus, the present disclosure shows that an irradiated, implantable biodiffusion chamber comprising freshly resected tumor cells and IGF-1R AS ODN safely serves as an effective, subject-specific autologous cell vaccine for cancer immunotherapy. As such, the use of the claimed implantable biodiffusion chamber to mount an immune response that selectively targets tumor cells in a subject provides a new and significant approach for the treatment of cancer, especially GBM.

Biodiffusion Chamber

A representative diffusion chamber comprises a chamber barrel having two ends, a first end and a second end. In embodiments, the biodiffusion chamber is a small ring capped on either side by a porous, cell-impermeable membrane, such as the Duropore membrane manufactured by Millipore Corporation. Optionally, one of the ends may be closed off as part of the chamber body leaving only one end open to be sealed using the porous membrane. The membranes can be made of plastic, teflon, polyester, or any inert material which is strong, flexible and able to withstand chemical treatments. The chamber can be made of any substance, such as and not limited to plastic, teflon, lucite, titanium, Plexiglass or any inert material which is non-toxic to and well tolerated by humans. In addition, the chambers should be able to survive sterilization. In some aspects, the diffusion chambers are sterilized with ethylene oxide prior to use. Other suitable chambers are described in U.S. Prov. No. 62/621,295, filed Jan. 24, 2018, U.S. Pat. No. 6,541,036, PCT/US16/26970, and U.S. Pat. No. 5,714,170, which are each incorporated herein by reference in their entirety.

In certain embodiments, the membrane allows passage of small molecules but does not allow passage of cells (i.e., the cells cannot leave or enter the chamber). In some aspects, the diameter of the pores of the membrane allows nucleic acids and other chemicals (such as, for example, cytokines produced by cells) to diffuse out of the chamber, does not allow passage of cells between the chamber and the subject in which it is implanted. The biodiffusion chambers useful in the present disclosure include any chamber which does not allow passage of cells between the chamber and the subject in which it is implanted, provided however, that the chamber permits interchange and passage of factors between the chamber and the subject. Thus, in certain aspects, the pore size has a cut-off that prevent passage of materials that are greater than 100 μm³ in volume into and out of the chamber. In some embodiments, the pores of the membrane have a diameter of about 0.25 μm or smaller. For example, the pores may have a diameter of about 0.1 μm. In particular aspects, the pores range in diameter from 0.1 pin to 0.25 μm. See also, Lange, et al., J. Immunol., 1994, 153, 205-211 and Lanza, et al., Transplantation, 1994, 57, 1371-1375, each of which is incorporated herein by reference in their entireties. This pore diameter prevents the passage of cells in or out of the chamber. In certain embodiments, diffusion chambers are constructed from 14 mm Lucite rings with 0.1 μm pore-sized hydrophilic Durapore membranes (Millipore, Bedford, Mass.).

In certain embodiments, a biodiffusion chamber comprises a membrane that allows the IGF-1R AS ODN to diffuse out of the chamber. In some embodiments, about 50% of the IGF-1R AS ODN diffuses out of the chamber in about 12 hours, about 60% of the IGF-1R AS ODN diffuses out of the chamber in about 24 hours, about 80% of the IGF-1R AS ODN diffuses out of the chamber in about 48 hours, and/or about 100% of the IGF-1R AS ODN diffuses out of the chamber in about 50 hours.

In an exemplary approach, to assemble the biodiffusion chamber, a first porous membrane is attached to one side of a first diffusion chamber, using glue and pressure to create a tight seal. A second porous membrane is similarly attached to a second diffusion chamber ring. The membranes can be secured in position with rubber gaskets which may also provide a tighter seal. The diffusion chamber rings are left overnight (minimum 8 hours) to dry. Then, the first diffusion chamber ring and the second diffusion chamber ring are attached to one another using glue and left overnight (minimum 8 hours) to dry. In a preferred embodiment, the first chamber ring and second chamber ring joining process comprises using 2 dichloroethane as a solvent to facilitate adhesion between the two rings. In an alternative approach, the chamber may have only one side that contains a porous membrane.

On the barrel portion of the chamber, one or more openings (e.g. ports) are provided which can be covered by a cap which is accessed from outside of the subject's body once the chamber is implanted, thus allowing the diffusion chamber to be refilled. The openings allow for multiple and sequential sampling of the contents, without contamination and without harming the subject, therefore significantly reducing the number of implantation procedures performed on the subject. Before implantation into the patient, the one or more openings may be sealed with bone wax, a port plug or cap made from, for example, PMMA. The cap can be a screw-on type of self-sealing rubber and fitted to the opening. In some configurations, the diffusion chamber may contain two or more injection openings or ports. Sampling of the chamber contents can be performed by accessing the opening by removing the cap on the outside of the subject's body and inserting an ordinary needle and syringe. In some embodiments, the chamber may further include a removal device. Such a device facilitates removal of the chamber from the patient.

In embodiments, the chamber serves as an antigen depot designed so that tumor antigens diffuse out of the chamber for the purpose of promoting a therapeutic host immune response. Exogenous IGF-1R AS ODN and ex vivo irradiation promote a pro-inflammatory response. This formulation is associated with clinical and radiographic improvements, prolonged survival on protocol, and represents a novel autologous cell vaccine that includes an exogenous active pharmaceutical ingredient (API) and radiation that we interpret as inducing or enhancing tumor immunity effect. Furthermore the addition of low concentration of the IGF-1R AS ODN is critical to a pro-inflammatory response.

In certain embodiments the disclosure provides a biodiffusion chamber for implantation into a subject suffering from cancer comprising: (a) tumor cells; and (b) an effective amount of an antisense molecule. In other embodiments is provided a method for treating cancer in a subject comprising: (a) obtaining a biodiffusion chamber comprising tumor cells and an effective amount of an antisense nucleic acid; (b) irradiating the biodiffusion chamber and contents; and (c) implanting the irradiated biodiffusion chamber into the subject for a therapeutically effective time.

In certain embodiments, the IGF-1R AS ODN is present in the biodiffusion chamber in an amount ranging from about 0.5 μg to about 10 μg. In certain aspects, the IGF-1R AS ODN is present in an amount ranging from about 1 μg to about 5 μg per chamber, or from about 2 μg to 4 μg per chamber. In some embodiments, the IGF-1R AS ODN is present in an amount of about 2 μg per chamber. In some embodiments, the IGF-1R AS ODN is present in an amount of about 4 μg per chamber. In some embodiments, is present in an amount of about 1.0 microgram (μg) to about 5.0 μg. For example, the IGF-1R AS ODN is present in an amount of about 1.0 μg, about 2.0 μg, about 3.0 μg, about 4.0 μg, about 5.0 μg, about 6.0 μg, about 7.0 μg, about 8.0 μg, about 9.0 μg, or about 10.0 μg per chamber. In some embodiments, the IGF-1R AS ODN is present in an amount of about 5.0 μg to about 50.0 μg per chamber. In some embodiments, the IGF-1R AS ODN is present in an amount of about 50.0 μg to about 100.0 μg per chamber. In some embodiments, the IGF-1R AS ODN is present in an amount of about 10.0 μg to about 500.0 μg per chamber. In some embodiments, the IGF-1R AS ODN is present in an amount of about 100.0 μg to about 500.0 μg per chamber. In some embodiments, the IGF-1R AS ODN is present in an amount of about 500.0 μg to about 1.0 milligram (mg) per chamber. In some embodiments, the IGF-1R AS ODN is present in an amount of about 1.0 mg to about 3.0 mg per chamber. In some embodiments, the IGF-1R AS ODN is present in an amount of about 3.0 mg to about 5.0 mg per chamber. In some embodiments, the IGF-1R AS ODN is present in an amount of about 5.0 mg to about 10.0 mg per chamber. In some embodiments, the IGF-1R AS ODN is present in an amount of about 1.0 μg to about 10.0 mg per chamber. Without being bound by theory it is thought that these levels promote an enhanced Th1 response in a subject, while avoiding an M2 immunostimulatory response in the subject.

In certain embodiments, the tumor cells are not treated with an IGF-1R AS ODN prior to encapsulation in the chamber. Typically, however, the tumor cells are treated with an IGF-1R AS ODN prior to encapsulation in the chamber. The time for treating the cells pre-encapsulation may vary. For example, the tumor cells may be treated ex vivo with an IGF-1R AS ODN immediately before encapsulation, for up to about 4 hours, for up to about 6 hours, for up to about 8 hours, for up to about 12 hours or for up to about 18 hours. Typically, the tumor tissue may be treated ex vivo for about 12 hours to about 18 hours pre-encapsulation. Conveniently, the cells may be encapsulated after a pre-treatment lasting up to overnight. Without being bound by theory, it is thought that the pre-encapsulation treatment plays a desirable role in stimulating production of tumor antigen.

The amount of IGF-1R AS ODN used for the pre-encapsulation treatment may be in a range of about 1 mg to 8 mg per million cells; for example, about 2 mg to about 6 mg per million cells, about 3 mg to about 5 mg per million cells. Typically the amount of IGF-1R AS ODN used for treatment prior to encapsulation is about 4 mg per million cells.

In some embodiments, the IGF-1R AS ODN for ex vivo treatment of the tumor cells is used at a concentration ranging from about at least 2 mg/ml to at least about 5 mg/ml. In certain aspects, the IGF-1R AS ODN is used at a concentration of at least 4 mg/ml. In specific embodiments, the IGF-1R AS ODN is used at a concentration of 4 mg/ml.

In certain embodiments, the IGF-1R AS ODN used to treat tumor cells ex vivo and the IGF-1R AS ODN present in the chamber are the same. In other embodiments, the IGF-1R AS ODN used to treat tumor cells ex vivo and the IGF-1R AS ODN present in the chamber are different. In certain embodiments, the IGF-1R AS ODN used to treat tumor cells ex vivo is at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, or at least about 50 nucleotides in length. In some embodiments, the IGF-1R AS ODN used to treat tumor cells ex vivo is from about 15 nucleotides to about 22 nucleotides in length. In certain aspects, the IGF-1R AS ODN used to treat tumor cells is about 18 nucleotides in length.

In certain embodiments, the IGF-1R AS ODN used to treat tumor cells ex vivo forms a secondary structure at 18° C., but does not form a secondary structure at about 37° C. In other embodiments, the IGF-1R AS ODN used to treat tumor cells does not form a secondary structure at about 18° C. or at about 37° C. In yet other embodiments, the IGF-1R AS ODN used to treat tumor cells ex vivo does not form a secondary structure at any temperature. In other embodiments, the IGF-1R AS ODN used to treat tumor cells does not form a secondary structure at 37° C. In particular embodiments, the secondary structure is a hairpin loop structure.

In some aspects, the IGF-1R AS ODN used to treat tumor cells comprises the nucleotide sequence of SEQ ID NO:1, or a fragment thereof. In certain embodiments, the IGF-1R AS ODN used to treat tumor cells may have at least about 70%, at least about 75%, at least about 80%, at least about 85%, at least about 90%, at least about 95%, at least about 96%, at least about 98%, or 100% identity to SEQ ID NO: 1, or a fragment thereof. In certain aspects, the IGF-1R AS ODN used to treat tumor cells is SEQ ID NO: 1.

After the tumor cells are treated with the AS-ODN for a period of time, the AS-ODN is removed and fresh AS-ODN is added to the chamber, which is then irradiated prior to implantation into a subject. In certain aspects, the biodiffusion chamber is treated with gamma irradiation at an amount of about 1 Gy, about 2 Gy, about 4 Gy, about 5 Gy, about 6 Gy, about 10 Gy, or up to about 15 Gy. In certain aspects, the dose of radiation is not more than about 5 Gy. In other aspects, the dose of radiation is at least about 5 Gy. In some aspects, the dose of radiation is 5 Gy. In certain embodiments, the biodiffusion chamber may be irradiated at least once, at least twice, at least three times, at least four times, or at least five times. In some embodiments, the chamber is irradiated less than about 24 hours prior to implantation into a subject. In other embodiments, chamber is irradiated about 24 hours prior to implantation into the subject. In yet other embodiments, the chamber is irradiated at least about 24 hours prior to implantation into the subject. In still other embodiments, the chamber is irradiated not more than about 48 hours prior to implantation into the subject. In yet other embodiments, the chamber is irradiated at least about 48 hours prior to implantation into the subject.

While the tumor cells are typically killed prior to implantation; for example by radiation, the cells need not be killed and indeed it may be advantageous to maintain the cells in an alive state to promote release of antigen. Thus, in certain embodiments, the cells may not be irradiated prior to implantation. For safety purposes, however, it is desirable to prevent release of live tumor cells into the subject.

Tumor cells can be placed in a diffusion chamber in varying numbers. In certain embodiments, about 1×10⁴ to about 5×10⁶ tumor cells are placed in each diffusion chamber. In other embodiments, about 1×10⁵ to about 1.5×10⁶ tumor cells are placed in the diffusion chamber. In yet other embodiments, about 5×10⁵ to about 1×10⁶ tumor cells are placed in the chamber, with a subject can be used. We have discovered that the number of tumor cells can impact the subjects' anti-tumor response and that an appropriate range should be selected to increase the chance to obtain the desired results. Patients implanted with 20 chambers had an anti-tumor immune response is optimal in a range of about 750,000 to about 1,250,000 cells in a chamber, with a peak at about 1 million cells/chamber. Multiple chamber containing irradiated tumor cells are administered and to maintain the optimal immune the response the number of cells/chamber is preferably maintained within the range. Preferably, the tumor cells are intact and not autolyzed or otherwise damaged as described herein.

In certain embodiments, it may be preferable to maintain the ratio of cells to AS ODN in a chamber. Thus, in certain aspects a chambers may contain about 2 μg of AS ODN and between 750,000 and 1,250,000 cells; for example 1,000,000 cells. The ratio of cells to AS ODN may thus be in a range from about 3.75×10⁵ to about 6.25×10⁵ per μg AS ODN; for example, about 5.0×10⁵ cells per μg. Thus, in a typical patient receiving 20 chambers the total dose of AS ODN is about 40 μg.

Typically, administration will be in a chamber as described herein; however, in certain aspects, the irradiated cells and IGF-1R AS ODN may be co-administered to the subject without being contained physically together in the chamber or another container. In certain methods using this approach, the irradiated cells IGF-1R AS ODN thus disperse, diffuse, or are metabolized in the body limited by the physiology of the subject. Thus, in certain aspects, e.g. the tumors cells for use may be prepared as described herein for the chamber and administered with the IGF-1R AS ODN but the administration may be not contained within a physical container. Such administration is typically intramuscular.

Tumor Tissue Preparation for Chamber

Tumor cells for use in the autologous vaccination are surgically removed from the subject. In embodiments, the tumor cells are removed from the patient using a tissue morselator. The extraction device preferably combines a high-speed reciprocating inner cannula within a stationary outer cannula and electronically controlled variable suction. The outer cannula has a diameter of 1.1 mm, 1.9 mm, 2.5 mm, or 3.0 mm, and a length of 10 cm, 13 cm, or 25 cm. The instrument also relies on a side-mouth cutting and aspiration aperture located 0.6 mm from the blunt desiccator end. The combination of gentle forward pressure of the aperture into the tissue to be removed and suction draws the desired tissue into the side aperture, allowing for controlled and precise tissue resection through the reciprocal cutting action of the inner cannula. A key feature is the absence of a rotation blade; this avoids drawing unintended tissue into the aperture. An example of a suitable device is the Myriad® tissue aspirator (NICO Corporation@ Indianapolis, Ind.), a minimally invasive surgical system which may be used for the removal of soft tissues with direct, microscopic, or endoscopic visualization. The shaved tissue is suctioned, gathered in to a collection chamber, and is collected in a sterile tissue trap. During collection of the tissue in the sterile tissue trap, blood is removed from the preparation. Preferably, the sterile trap contains a collection dish at the bottom of the trap and a stem that provides access to the trap. The trap structure may also contain an inner ladle-shaped structure that is removable from the trap to facilitate tissue removal from the trap.

Preferably, the morselator generates no heat at the resection site or along its shaft, and requires no ultrasonic energy for tissue removal. Thus, in particular embodiments, the tumor tissue is morselized tumor tissue (i.e. tumor shaved tissue obtained by side-mouth cutting in the absence of heat, and optionally in the absence of ultrasonic treatment). Advantageously, the aspirator-extract and morselized tissue has higher viability than tissue removed by other methods. It is believed that the extraction process maintains higher tumor cell viability in part due to restricting exposure of the tumor cells to high temperatures during removal. For example, the methods herein do not expose tumor cells to above 25° C. during removal. Thus, the cells are not exposed to temperatures above body temperature, i.e., about 37° C.

The amount of tumor tissue obtained from the subject may vary. Preferably, the amount is at least 1, at least 2, at least 3 grams or at least 4 grams of wet tumor tissue is obtained from the patient. The tissue is removed from the sterile tissue trap and disaggregated by pipetting with a sterile pipette to break up large tissue fragments. The disaggregated cell suspension is then placed onto sterile tissue culture plates in serum-containing media, and incubated in a tissue culture incubator. This plating step serves to enrich the desired functional cells by adherence, and also helps to remove debris from the preparation. Thus, the tumor cells used in treatments described herein preferably consist essentially of, or consist of, adherent cells from the tumor tissue.

After a predetermined incubation time (e.g., 6, 12, 24, or 48 hours), the cells are removed from the plates. The cells may be removed by scraping, by chemical methods (e.g. EDTA) or by enzymatic treatment (e.g. trypsin). The cells are placed into one or more diffusion chambers. In some embodiments, the cells are split between 2, 3, 4, 5, 6.7, 8, 9, 10, 11, 12, 13, 14, 15, 16, 17, 18, 19, 20, 21, 22, 23, 24, 25, 26, 27, 28, 29, 30 or more diffusion chambers. Often, 20 chambers are used. In some embodiments, each diffusion chamber contains an equal number of cells. In some embodiments, a first diffusion chamber contains more cells than a second chamber.

In some embodiments, the cells are sorted before being placed in the chamber. In some embodiments, the cells are enriched by selecting for one or more cellular markers before being placed in the chamber. The selection may be performed, for example, using beads or by cell sorting techniques known to those of skill in the art. In some embodiments, the cells placed into the chamber are enriched for one or more markers.

In some embodiments, implantation of the biodiffusion chamber for a therapeutically effective time reduces or eliminates return of the cancer in the subject. In certain aspects, implantation of the biodiffusion chamber causes a reduction of tumor volume associated with the cancer in the subject. In yet other embodiments, implantation of the biodiffusion chamber for a therapeutically effective time induces elimination of the tumor in the subject. In some embodiments, implantation of the chamber inhibits regrowth of the tumor for at least 3 months, at least 6 months, at least 12 months, at least 36 month, or indefinitely.

The biodiffusion chamber can be implanted in a subject in the following non-limiting ways: subcutaneously, intraperitoneally, and intracranially. In certain embodiments, the diffusion chamber(s) is implanted into an acceptor site of the body having good lymphatic drainage and/or vascular supply such as the rectus sheath. In other embodiments, a refillable chamber can be employed such that the diffusion chamber can be re-used for treatments and emptied following treatments. In certain aspects, a plurality of diffusion chambers, preferably between 5 and 20, can be used in a single subject.

In certain embodiments, at least about 1, at least about 2, at least about 3, at least about 4, at least about 5, at least about 10, at least about 15, at least about 20, at least about 25, at least about 30, at least about 35, at least about 40, at least about 45, or at least about 50 chambers are implanted into the subject. In some embodiments, 10-20 chambers are implanted into the subject. Preferably, about 20 chambers are implanted into the subject. In certain embodiments, the tumor cells are divided equally among each chamber.

Typically, the chamber is removed after period of time. For example, the chamber may be implanted in the subject for about 24 hours, about 48 hours, about 72 hours, or about 96 hours. Implantation for about 48 hours is associated with beneficial therapeutic outcomes. Accordingly, the preferred time of implantation is about 48 hours. In certain embodiments, the vaccination procedure is performed one time per patient. In other embodiments, the vaccination procedure is performed multiple times per patient. In embodiments, the vaccination procedure is performed two times, three times, four times, five times, six times, seven times, or eight times in a single patient. In embodiments, the vaccination is repeated every 7, 14, or 28 days, or every 1, 3, or 6 months for a given period of time. In further embodiments, the vaccination procedure is repeated periodically until the patient is free of cancer.

Without being bound by theory, it is thought that implantation of the biodiffusion chamber causes elimination or reduction of M2 cells at or near the implantation site such that an immune response against tumor antigens diffusing out from the chamber is achieved. In certain aspects, elimination or reduction of M2 cells at the implantation site leads to enhanced presentation of autologous tumor antigens by antigen-presenting cells (APC) to CD4 T cells leading to production of interferon-gamma (IFNγ) and the induction of type 1 tumor immunity. In certain aspects, the production of IFNγ by tumor antigen-specific CD4 T cells and the anti-M2 effects of IGF-1R AS ODN drive type 1 anti-tumor immunity and the loss of anti-inflammatory M2 cells from the circulation and tumor microenvironment indirectly interfering with tumor growth. In some aspects, the production of IFNγ by tumor antigen-specific CD4 T cells and the anti-M2 effects of IGF-1R AS ODN unleashes effector-mediated damage to the tumor cells and tumor microenvironment (M2 cells) and initiates the longer process of programming memory T cells recognizing tumor antigens. In certain embodiments, the anti-tumor adaptive immune response sustains continued tumor regression.

Optionally, the cells introduced into the chamber may be enriched for certain cell types. Nestin a, cytoskeleton-associated class VI intermediate filament (IF) protein, has traditionally been noted for its importance as a neural stem cell marker. We have discovered that in certain brain tumor samples, cells positive for nestin (nestin+ cells) are enriched compared to benign tissue, and that this associated corresponds to improved therapeutic response. Thus, in certain aspects, a subject's tumor can be biopsied to assess the degree of nestin expression, and therefore, in certain aspects, the chamber cells are enriched Nestin-positive (“+”) cells compared to benign tissue. Without being bound by theory, it is thought that nestin provides a marker associated with antigens suitable useful in producing an anti-tumor immune response. Accordingly, the cells implanted into the chamber may be enriched for nestin+ cells compared to the tumor cell population as a whole when extracted from the subject. An enhanced immune response is in some embodiments obtained when the tumor sample used to stimulate a response is enriched with Nestin.

Systemic Administration

As an alternative to, or supplement to, implantation of the chambers, IGF-1R AS ODN may be administered systemically. Thus, in embodiments, the IGF-1R AS ODN is provided in a pharmaceutical composition for systemic administration. In addition to the IGF-1R AS ODN, the pharmaceutical composition may comprise, for example, saline (0.9% sodium chloride). The composition may comprise phospholipids. In some aspects, the phospholipids are uncharged or have a neutral charge at physiologic pH. In some aspects, the phospholipids are neutral phospholipids. In certain aspects, the neutral phospholipids are phosphatidylcholines. In certain aspects, the neutral phospholipids are dioleoylphosphatidyl choline (DOPC). In some aspects, the phospholipids are essentially free of cholesterol.

In some aspects, the phospholipids and oligonucleotides are present at a molar ratio of from about 5:1 to about 100:1, or any ratio derivable therein. In various aspects, the phospholipids and oligonucleotides are present at a molar ratio of about 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, or 100:1. In some aspects, the oligonucleotides and phospholipids form an oligonucleotide-lipid complex, such as, for example, a liposome complex. In some aspects, at least 75%, 76%, 77%, 78%, 79%, 80%, 81%, 82%, 83%, 84%, 85%, 86%, 87%, 88%, 89%, 90%, 91%, 92%, 93%. 94%, 95%, 96%, 97%, 98%, or 99% of the liposomes are less than 5 microns in diameter. In various aspects, the composition further comprises at least one surfactant, such as, for example, polysorbate 20. In some aspects, at least about 5% of the total liposomal antisense drug product consists of surfactant and at least about 90% of the liposomes are less than 5 microns in diameter. In some aspects, at least about 15% of the total liposomal antisense drug product consists of surfactant and at least about 90% of the liposomes are less than 3 microns in diameter. In some aspects, the population of oligonucleotides are incorporated in the population of liposomes.

In some aspects the pharmaceutical composition is a liquid pharmaceutical composition. In other aspects, the pharmaceutical composition is a solid pharmaceutical composition.

Dosages for systemic administration of the antisense in human subjects may be about 0.025 g/kg, about 0.05 g/kg, about 0.1 g/kg, about 0.15 g/kg, or about 0.2 g/kg. In certain embodiments, the dosage for systemic administration may be from 0.025 g/kg to 0.2 g/kg. In some embodiments, the dosage is about 0.2 g/kg. In other embodiments, the dosage is from 0.004 g/kg to 0.01 g/kg. In other embodiments, the dosage is less than 0.01 g/kg. In further embodiments, the dosage is not between 0.01 g/kg to 0.2 g/kg. In certain aspects, the antisense is supplied as a lyophilized powder and re-suspended prior to administration. When resuspended the concentration of the antisense may be about 50 mg/ml, about 100 mg/ml, about 200 mg/ml, about 500 mg/ml, about 1000 mg/ml, or a range between those amounts.

In certain embodiments, the AS ODN may be administered systemically pre-operatively; for example prior to surgery to reduce tumor burden. For example, the AS ODN may be administered up to 24 hours, up to 36 hours, up to 48 hours or up to 72 hours before surgery. In particular aspects, the pharmaceutical composition may be administered about 48 to about 72 hours before surgery. Typically, in such circumstances, the administration is by intravenous bolus.

Combination Therapies

Historically, cancer therapy has involved treating subjects with radiation, with chemotherapy, or both. Such approaches have well-documented challenges. Advantageously, however, the chamber implantation methods disclosed herein may be used to treat a subject having cancer as a monotherapy. Thus it is preferable that the methods disclosed herein do not include chemotherapy or radiation therapy. Notwithstanding the excellent effect achieved by monotherapy approaches herein, however, it may be beneficial under certain circumstances to combine the chamber methods with other therapies; for example, radiation therapy. In certain embodiments, the radiation therapy includes, but is not limited to, internal source radiation therapy, external beam radiation therapy, and systemic radioisotope radiation therapy. In certain aspects, the radiation therapy is external beam radiation therapy. In some embodiments, the external beam radiation therapy includes, but is not limited to, gamma radiation therapy, X-ray therapy, intensity modulated radiation therapy (IMRT), and image-guided radiation therapy (IGRT). In certain embodiments, the external beam radiation therapy is gamma radiation therapy. Radiation may be administered before chamber implantation or after implantation; for example, as a salvage therapy. Typically, such salvage therapy approaches are not implemented until the cancer is determined to have returned.

Thus, in certain combination approaches, both the chamber methods, and the systemic methods and compositions, described herein may be used in the same subject, alone or in combination with radiation or chemotherapy. In the combination approaches described herein, the chamber implantation is preferably used as a first-line therapy. Using the chamber implantation first is desirable because the subject's immune system can be inhibited by other therapies, reducing the therapeutic benefit of the chamber implantation.

Optionally, systemic administration may be performed prior to chamber implantation. Such an approach can be used to enhance the subjects immune system, as a priming approach. The priming approach may be especially advantageous where prior therapy has resulted in the subject having a compromised immune system.

When systemic administration is used in combination, the AS ODN may be systemically administered at least 2 weeks, at least 1 week, at least 3 days, or at least 1 day prior to treatment of the patient using an autologous cancer cell vaccine. In other embodiments, the AS ODN may be systemically administered at least 1 day, at least 3 days, at least 1 week, or at least 2 weeks following treatment of the patient using an autologous cancer cell vaccine; i.e. the chamber.

Optionally, the subject may be revaccinated with chambers using the methods described here subsequent to the first vaccination. A second or further additional vaccination may use tumor cells taken from the subject during the tissue removal and stored. Optionally, the second or further additional vaccination may use fresh tumor tissue removed from the subject and treated as described herein. Any tumor remaining in the subject may express the same antigens and thus act as a depot, providing for re-stimulation. However, recurring tumors may develop new antigens and thus provide additional options to stimulate an anti-tumor response. A subsequent vaccination may be after the first treatment is complete and the tumor has recurred or if the subject has not responded to the first treatment.

Subjects for Treatment with the IGF-1R AS ODN

Suitable subjects are animal with cancer; typically, the subject is a human. While brain cancers, such as glioblastoma, benefit particularly from the methods disclosed herein, the methods apply to cancer generally. Accordingly, the disclosure provides methods of treating cancers, including those selected from the group consisting of: glioma, astrocytoma, hepatocarcinoma, breast cancer, head and neck squamous cell cancer, lung cancer, renal cell carcinoma, hepatocellular carcinoma, gall bladder cancer, classical Hodgkin's lymphoma, esophageal cancer, uterine cancer, rectal cancer, thyroid cancer, melanoma, colorectal cancer, prostate cancer, ovarian cancer, and pancreatic cancer. In specific embodiments, the cancer is a glioma. In certain aspects, the glioma is recurrent malignant glioma. In some embodiments, the cancer is an astrocytoma. In certain embodiments, the subject who is a candidate for treatment is suffering from WHO grade H, WHO grade III, or WHO grade IV tumor. In some aspects, the tumor is an astrocytoma. In certain embodiments, the tumor is selected from grade II astrocytoma, AIII (IDH1R132H mutant grade HI astrocytoma), AIII-G (IDH1 wild-type grade HI with characteristics of glioblastoma multiforme astrocytoma), or grade IV astrocytoma.

Grade IV astrocytoma is the highest grade glioma and is synonymous with glioblastoma (GBM). With a yearly incidence of 3 or 4 per 100,000 GBM is the most common malignant primary brain tumor in adults. Standard of care therapy—typically a combination of radiotherapy and chemotherapy using Temozolomide—does not work well and the outcome of GBM patients remains poor with a median life expectancy of 15-17 months. Advantageously, the methods here may be used to treat newly diagnosed brain cancers and may also be used to treat recurrent glioblastoma; for example, in patients previously treated with standard of care therapy. Thus, in certain aspects, the subject may be a newly diagnosed GBM subject or a recurrent GBM subject. The subject is preferably one who has not been previously treated with any therapeutic approaches that are immunosuppressive. In particular aspects, eligible subjects are over 18 years of age and have a Karnofsky score of 60 or above. Optionally, the subjects do not have bihemispheric disease and/or do not have an autoimmune disease.

Optionally, a subject who is a candidate for treatment may be identified by performing a tumor biopsy on the subject. In some embodiments, tumors from the subject are assayed for the presence of monocytes. In certain aspects, the monocytes include, but are not limited to, CD11b+, CD14+, CD15+, CD23+, CD64+, CD68+, CD163+, CD204+, or CD206+ monocytes. The presence of monocytes in the tumors may be assayed using immunohistochemistry. In certain embodiments, a subject who is a candidate for treatment shows CD163+M2 cells greater than about 10%, about 15%, about 20%, about 25%, about 30%, about 35%, about 40%, about 45%, or about 50% of the subjects total peripheral blood mononuclear cells (PBMCs). In certain aspects, the subject shows CD163+M2 cells greater than about 20% of the subject's total PBMCs.

In yet other embodiments, a subject who is a candidate for treatment is identified by the presence of one or more cytokines in the serum of the subject. These cytokines include, without limitation, CXCL5, CXCL6, and CXCL7, IL6, IL7, IL8, IL10, IL11, IFN-γ, and HSP-70.

In yet other embodiments, a subject who is a candidate for treatment is identified by the presence of one or more growth factors in the serum of the subject. These growth factors include, without limitation, FGF-2, G-CSF, GM-CSF, and M-CSF.

In some embodiments, a subject who is a candidate for treatment with the biodiffusion chamber is identified by measuring the levels of a specific set of cytokines. In some embodiments, the subject has elevated levels of these cytokines in comparison to a healthy subject. As used herein, the term “healthy subject” refers to a subject not suffering from cancer or any other disease and not in need of treatment with the biodiffusion chamber.

In particular embodiments, the cytokines may be added to the chamber to augment the anti-tumor immune response. For example, the cytokines added to the chamber may be selected from the group consisting of CCL19, CCL20, CCL21, and CXCL12, and combinations thereof.

In certain embodiments, the circulating CD14+ monocytes have an elevated level of CD163 in comparison to a healthy subject. In some aspects, the levels of CD163 on the circulating CD14+ monocytes are elevated by at least about 2 fold, at least about 3 fold, at least about 4 fold, at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, or at least about 100 fold in comparison to a healthy subject. In particular embodiments, the levels of CD163 on the circulating CD14+ monocytes are elevated by about 2 fold in comparison to a healthy subject.

In other embodiments, a subject who is a candidate for treatment has serum that polarizes undifferentiated monocytes towards M2 cells. In certain aspects, incubation of the subject's sera with undifferentiated monocytes induces the expression of one or more cell surface markers on the monocytes including, but not limited to, CD11b, CD14, CD15, CD23, CD64, CD68, CD163, CD204, and/or CD206. In other aspects, incubation of the subject's sera with undifferentiated monocytes elevates the expression of one or more cell surface markers on the monocytes in comparison to monocytes not incubated with the subject's sera. In certain aspects, the cell surface markers include, but are not limited to, CD11b, CD14, CD15, CD23, CD64, CD68, CD163, CD204, and/or CD206. In some aspects, the levels of one or more surface markers are elevated by at least about 1.3 fold, at least about 1.5 fold, at least about 1.8 fold, at least about 2 fold, at least about 3 fold, at least about 4 fold, at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, or at least about 100 fold in comparison to undifferentiated monocytes not incubated with the subject's sera. In particular embodiments, the levels of one or more surface markers are elevated by about 2 fold in comparison to undifferentiated monocytes not incubated with the subject's sera. Monocytes polarized by a subject's sera may be measured using FACS.

Target Cells

Without being bound by theory it is thought that the AS ODN reduces the subjects M2 cells and/or inhibits polarization of cells into M2 cells by downregulating IGF-1R expression. In some embodiments, IGF-1R expression in M2 cells is downregulated by at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% in comparison to cells not treated with the antisense. IGF-1R expression in M2 cells may be measured by quantitative RT-PCR.

In some embodiments, IGF-1R expression in M2 cells remains downregulated in the subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 3 weeks, at least about 4 weeks, at least about 5 weeks, or at least about 6 weeks after receiving one dose of the antisense.

In some aspects, the downregulation of expression of IGF-1R in M2 cells causes a selective reduction of M2 cells in a subject in comparison to cells not expressing IGF-1R. In certain embodiments, M2 cells in a subject are reduced by at least about 2%, at least about 5%, at least about 10%, at least about 20%, at least about 30%, at least about 40%, at least about 50%, at least about 60%, at least about 70%, at least about 80%, at least about 90%, or at least about 95% in comparison to a subject not treated with the antisense. In other embodiments, the M2 cell population is eliminated. For example, after implantation of the biodiffusion chamber, the M2 cell population may be about 1%, about 2%, about 5%, or about 10% of the population before implantation of the biodiffusion chamber. M2 cells in a subject may be measured using FACS. In certain aspects, after treatment the M2 cells are eliminated; i.e., undetectable by FACS. In other aspects, the decrease in M2 cells may be measured using a proxy assay; for example, serum from the subject may be obtained before and after treatment to assess its ability to polarize M2 cells. Following treatment with methods disclosed herein, the ability of the serum to polarize M2 cells is reduced by about 80% to about 100%, about 20% to about 60%, or about 10% to about 50%.

In some embodiments, targeting the expression of IGF-1R in M2 cells causes the M2 cells to undergo cell death. In certain embodiments, the cell death is necrosis. In other embodiments, the cell death is apoptosis. Apoptosis, for purposes of this disclosure, is defined as programmed cell death and includes, but is not limited to, regression of primary and metastatic tumors. Apoptosis is a programmed cell death which is a widespread phenomenon that plays a crucial role in the myriad of physiological and pathological processes. Necrosis, in contrast, is an accidental cell death which is the cell's response to a variety of harmful conditions and toxic substances. In yet other embodiments, targeting the expression of IGF-1R in M2 cells causes the M2 cells to undergo cell cycle arrest.

Kits

Preparation of a completed chamber requires multiple components and multiple steps. In another aspects of the disclosure kits containing components for practicing the methods disclosed herein are provided. In certain aspects, the kits comprise the chamber body, which may be present in one portion or in two halves. Items to seal the chamber may also be included including one or more membranes, glues and solvents (e.g., an alcohol, or 2 dichloroethane). Optionally, the membrane may by sonically welded onto the chamber to create a seal. The kits include the antisense ODN. Optionally, the ODN may be divided into two portions. A first portion to treat the cells after surgical removal from the subject, and a second portion to combine with the cells when introduced into the subject. Other optional kit items include media for culturing the cells, and antibiotics for preventing bacterial growth in the media.

Optionally, chambers in the kit may be pre-connected (e.g by suture) to each other using an eyelet or other device attached to the chamber and adapted to receive the connecting material. Advantageously, by pre-connecting multiple chambers, the desired number of chambers may be readily introduced and removed by the surgeon.

In addition to various aspects and embodiments disclosed herein the following embodiments are specifically contemplated:
1. A method comprising determining MGMT methylation and/or T-cell function in a subject having cancer and administering to said subject an IGF-1R AS ODN.
2. A method comprising determining MGMT methylation in a subject having cancer and administering to said subject an IGF-1R AS ODN.
3. A method comprising determining MGMT methylation in a subject having cancer and administering to said subject an IGF-1R AS ODN only if said subject has methylated MGMT.
4. A method comprising determining the T-cell function in a subject having cancer and administering to said subject an IGF-1R AS ODN.
5. A method comprising determining the T-cell function in a subject having cancer and administering to said subject an IGF-1R AS ODN only if said subject has good T-cell function.
6. A method comprising identifying a subject having cancer and having an increased likelihood of responding to an IGF-1R AS ODN and administering to said subject an IGF-1R AS ODN.
7. A method comprising identifying a subject having cancer and having an increased likelihood of responding to an IGF-1R AS ODN and administering to said subject an IGF-1R AS ODN; wherein said increased likelihood of responding to an IGF-1R AS ODN is evaluated by determining MGMT methylation and/or T-cell function in said subject.
8. A method comprising identifying a subject having cancer and having an increased likelihood of responding to an IGF-1R AS ODN and administering to said subject an IGF-1R AS ODN; wherein said increased likelihood of responding to an IGF-1R AS ODN is evaluated by determining MGMT methylation and/or T-cell function in said subject; and wherein said increased likelihood is established by identifying MGMT methylation in said subject and/or by determining good T cell function in said subject.
9. A method comprising identifying a subject having cancer and having an increased likelihood of responding to an IGF-1R AS ODN and administering to said subject an IGF-1R AS ODN; wherein said increased likelihood of responding to an IGF-1R AS ODN is evaluated by determining MGMT methylation; and wherein said increased likelihood is established by identifying MGMT methylation in said subject.
10. A method comprising identifying a subject having cancer and having an increased likelihood of responding to an IGF-1R AS ODN and administering to said subject an IGF-1R AS ODN; wherein said increased likelihood of responding to an IGF-1R AS ODN is evaluated by determining T-cell function in said subject; and wherein said increased likelihood is established by determining good T cell function in said subject.
11. A method of predicting the prognosis of a subject having cancer in response to an IGF-1R AS ODN; said method comprising determining the MGMT methylation and/or determining the T-cell function in said subject.
12. A method of predicting the prognosis of a subject having cancer in response to an IGF-1R AS ODN, said method comprising determining the MGMT methylation and/or determining the T-cell function in said subject; wherein methylated MGMT and/or good T cell function in said subject is indicative of a favorable prognosis.
13. A method of predicting the prognosis of a subject having cancer in response to an IGF-1R AS ODN, said method comprising determining the MGMT methylation in said subject; wherein methylated MGMT in said subject is indicative of a favorable prognosis.
14. A method of predicting the prognosis of a subject having cancer in response to an IGF-1R AS ODN, said method comprising determining the T-cell function in said subject; wherein good T cell function in said subject is indicative of a favorable prognosis.
15. A method of predicting the prognosis of a subject having cancer in response to an IGF-1R AS ODN, said method comprising determining the MGMT methylation and/or determining the T-cell function in said subject; wherein unmethylated MGMT and/or poor T cell function in said subject is indicative of an unfavorable prognosis.
16. A method of predicting the prognosis of a subject having cancer in response to an IGF-1R AS ODN, said method comprising determining the MGMT methylation in said subject; wherein unmethylated MGMT in said subject is indicative of an unfavorable prognosis.
17. A method of predicting the prognosis of a subject having cancer in response to an IGF-1R AS ODN, said method comprising determining the T-cell function in said subject; wherein poor T cell function in said subject is indicative of an unfavorable prognosis.
18. The method of any of the preceding embodiments wherein said T cell function is determined by evaluating the number of expressing IFN-γ in response to nonspecific stimulation.
19. The method of any of the preceding embodiments wherein said T cell function is determined by evaluating the number of expressing IFN-γ in response to nonspecific stimulation; and wherein a median or greater number of T cells expressing IFN-γ in response to nonspecific stimulation is classified as good T cell function and less than median or lessor number of T cells expressing IFN-γ in response to nonspecific stimulation is classified as poor T cell function.
20. The method of any of the preceding embodiments wherein said IGF-1R AS ODN is administered to subject before temozolamide is administered to said subject.
21. The method of any of the preceding embodiments wherein said IGF-1R AS ODN is administered to subject at least 2 weeks; at least 3 weeks; least 4 weeks; at least 5 weeks; at least 6 weeks; at least 7 weeks; or at least 8 weeks before temozolamide is administered to said subject.
22. The method of any of the preceding embodiments wherein the IGF-1R AS ODN is administered to the subject as an autologous cancer cell vaccine.
23. The method of any of the preceding embodiments wherein the IGF-1R AS ODN is administered to the subject as a fully formulated biodiffusion chamber.
24. The method of any of the preceding embodiments wherein the biodiffusion chamber, if present is prepared by: (a) encapsulating tumor cells obtained from the subject into the biodiffusion chamber in the presence of an IGF-1R AS ODN, wherein the ratio of tumor cells to IGF-1R AS ODN in the chamber is in a range from about 3.75×10⁵: 1 □g to about 6.25×10⁵: 1 μg; wherein the tumor cells are obtained from the subject using a tissue morselator, and (b) irradiating the biodiffusion chamber.
25. The method of any of the preceding embodiments that involve a biodiffusion chamber, wherein the tumor cells are enriched for nestin expression before they are placed into the biodiffusion chamber.
26. The method of any of the preceding embodiments that involve a biodiffusion chamber, wherein the tumor cells in the chamber are enriched for adherent cells compared to the tumor cells obtained from the subject.
27. The method of any of the preceding embodiments that involve a biodiffusion chamber, wherein the tumor cells consist essentially of adherent cells.
28. The method of any of the preceding embodiments that involve a biodiffusion chamber, wherein the cells are treated with IGF-1R AS ODN before encapsulation into the chamber.
29. The method of any of the preceding embodiments that involve a biodiffusion chamber, wherein the IGF-1R AS ODN is present at about 2 mg to about 6 mg per million cells during the treatment before encapsulation.
30. The method of any of the preceding embodiments that involve a biodiffusion chamber wherein the IGF-1R AS ODN is present at about 4 mg per million cells during the treatment before encapsulation.
31. The method of any of the preceding embodiments that involve a biodiffusion chamber, wherein the treatment with IGF-1R AS ODN prior to encapsulation is for up to about 18 hours.
32. The method of any of the preceding embodiments that involve a biodiffusion chamber, wherein the treatment with IGF-1R AS ODN prior to encapsulation is for about 12 hours to about 18 hours.
33. The method of any of the preceding embodiments, wherein the IGF-1R AS ODN has the sequence of SEQ ID NO:1.
34. The method of any of the preceding embodiments that involve a biodiffusion chamber, wherein the IGF-1R AS ODN in the chamber is present at about 2 μg.
35. The method of any of the preceding embodiments that involve a biodiffusion chamber, wherein the irradiated tumor cells are present in a range from about 750,000 to about 1,250,000 per chamber.
36. The method of any of the preceding embodiments that involve a biodiffusion chamber, wherein the irradiated tumor cells are present at about 1,000,000 per chamber.
37. The method of any of the preceding embodiments that involve a biodiffusion chamber, comprising implanting two or more biodiffusion chambers into the subject.
38. The method of any of the preceding embodiments that involve a biodiffusion chamber; wherein about 10 to about 30 biodiffusion chambers are implanted into the subject.
39. The method of any of the preceding embodiments that involve a biodiffusion chamber, wherein about 10 to about 20 biodiffusion chambers are implanted into the subject.
40. The method of any of the preceding embodiments that involve a biodiffusion chamber, wherein the diffusion chambers are implanted into the subject for 48 hours.
41. The method of any of the preceding embodiments, wherein the cancer is a brain cancer.
42. The method any of the preceding embodiments, wherein the brain cancer is selected from a grade II astrocytoma, a grade AIII astrocytoma, a grade AIII-G astrocytoma, and a grade IV astrocytoma (glioblastoma multiforme).
   The method of any of the preceding embodiments, wherein the brain cancer is a grade IV astrocytoma (glioblastoma multiforme).
43. The method of any of the preceding embodiments wherein said subject is a human.

### EXAMPLES

### Example 1

Introduction

We evaluated IGV-001, a unique combination vaccine, in adults with newly diagnosed glioblastoma. IGV-001 consists of autologous glioblastoma tumor cells and an antisense oligodeoxynucleotide against insulin-like growth factor type 1 receptor (IGF-1R) DNA/mRNA (IMV-001; previously designated NOBEL), co-administered via biodiffusion chambers implanted in the abdomen. IGV-001 is believed to promote tumor immunity through the release of tumor antigen with concomitant stimulation of antigen presentation. This Phase 1b study builds on our Phase 1a study in patients with recurrent, World Health Organization grade III or IV astrocytomas, in which 8 of 12 patients showed radiographic improvement.

Methods

Study Design

Adults with radiographically confirmed, newly diagnosed malignant glioma were enrolled. A Karnofsky Performance Status score of at least 60, was also required. Patients were randomized to one of 4 IGV-001 exposures: lowest (10 chambers implanted for 24 hours); lower (10 chambers implanted for 48 hours); higher (20 chambers implanted for 24 hours); and highest (20 chambers implanted for 48 hours).

During craniotomy for tumor resection, the Myriad tissue aspirator was utilized to aspirate and comminute the tumor tissue and maintain the viability of the tumor cells according to standard of care (SOC). The surgeon created an abdominal acceptor site between the rectus sheath and the rectus abdominis muscle for subsequent implantation of diffusion chambers. Harvested tumor cells were treated ex vivo with IMV-001 for 4-8 hours, then encapsulated in 10 or 20 1.4 cm biodiffusion chambers (depending on randomization) with additional IMV-001 and irradiated with 5 Gy. Radiation of IMV-001-treated cells causes their release of immunostimulatory antigens. Chambers were implanted in the abdominal acceptor sites within 24 hours of craniotomy. Chambers were removed after 24 or 48 hours (depending on randomization), and the abdomen closed. Because previous experience with IMV-001 yielded a higher than expected incidence of deep vein thrombosis (DVT), prophylactic enoxaparin was administered daily for 3 months and patients were monitored for DVT by compression ultrasound twice weekly during initial hospitalization, then monthly for 3 months. SOC (ie, radiation and temozolomide [TMZ]) was initiated 4-6 weeks post-surgery, for a duration of 6 weeks. Patients received another 12 cycles of TMZ as maintenance treatment.

Because the highest exposure cohort showed the highest levels of circulating, potentially therapeutic, pro-inflammatory cytokines after therapy, as well as clinical and radiographic improvements, randomization halted after patient 23. The protocol was amended, and subsequent patients received the highest exposure. This amendment reflected a shift from a protocol with the primary objective of documenting safety with associated exploratory biomarker evaluations, to a protocol with clinical exploratory end points including overall survival (OS) and progression-free survival (PFS).

Assessments

Adverse events (AEs) and serious AEs were recorded from chamber implantation until 30 days post study exit, for a minimum of 6 weeks post treatment. AEs were assessed and categorized according to the National Cancer Institute Common Toxicity Criteria for Adverse Events v4.03. Asymptomatic grade 1 and grade 2 laboratory values were not captured as AEs unless determined to be clinically significant by the treating physician. Magnetic resonance imaging (MRI) was performed within 14 days prior to surgery, and at post-operative timepoints up to at least 24 months. Radiographic interpretations of MRI scans were performed by neuroradiologists blinded to patients' corticosteroid dosage and clinical status. Radiographic responses were based on Response Assessment in Neuro-Oncology (RANO)2 and immunotherapy RANO (iRANO) criteria. Time to progression was assessed from date of surgery to the date of the first observation of objective disease progression measured by MRI. Evidence of disease progression was required to be corroborated by an independent radiology review committee. PFS was measured from the date of surgery to progression or censoring (censoring refers to the exclusion of a patient from the clinical study for any of various criteria). OS was the time elapsed between the date of surgery and latest follow-up or death. Patients considered withdrawn from the study were followed for OS.

Because the mechanism of action of IGV-001 is believed to rely heavily on the immune response, we quantified circulating lymphocyte and monocyte subsets, serum cytokines and chemokines, and T cell function (based on number of T cells expressing the pro-inflammatory cytokine interferon-γ [IFN-γ] in response to nonspecific stimulation) before and after treatment.

Statistical Analysis

The intent-to-treat (ITT) population included all enrolled patients that were not screen failures and was used for evaluation of safety and clinical outcomes. The number and percentage of subjects with AEs were summarized overall, by severity grade, and by association with investigational agent or SOC and preferred term. For multiple AEs per subject within a preferred term, only the most severe is reported. Time-to-event data (PFS and OS) were analyzed using the product-limit method and graphed with points connected using a step function. SAS version 9.4 (SAS Institute; Cary, N.C.) was used for all analyses. Using the method described by Guyot et al (2012), patient-level data from the SOC arms of published studies with similar enrolment criteria were estimated. OS and PFS for these SOC arms were compared to our IGV-001-treated cohort using the log-rank test.

T Cell Assay Protocol

Whole blood or leukopheresis samples obtained from patients in the glioblastoma clinical trial were separated and the PBMC were cryopreserved in DMSO. These PBMC samples were utilized for isolation and stimulation of T cells. Downstream analysis of the triggered cells was performed using ELISPOT.

Isolation of Naïve T Cells

The PBMC samples were thawed and washed with warm RPMI media supplemented with 10% FBS, 1% L-Glutamine, 1% Penicillin/Streptomycin to remove DMSO. The pellet was re-suspended in a known volume of media and an initial cell count was obtained using Countess II FL automated cell counter (ThermoFisher Scientific). Naive T cell isolation using magnetic beads was performed by a negative selection method using Easysep Human T cell isolation kit (Stem cells technology #17951). T cells isolation was performed simultaneously on multiple samples from the clinical trial in a 96 well plate.

In-vitro stimulation of T cells—ELISPOT Assay

Polyvinylidene difluoride membrane Elispot plates (96 well plate, Merck Millipore #S2EM004M99) were coated with anti-IFN-γ monoclonal antibody (Mabtech #3420-3-1000) and incubated overnight for 14-16 hours at 4° C. The antibody solution was discarded and the plates blocked using serum-free media. After 2 hours of incubation at room temperature, media was decanted and 50,000 T cells were added into each well along with Anti. CD3/CD28/CD2 (Immunocult, Stem cells Technologies #10910) T cell activator and incubated for 20 hours at 37° C., 5% CO2. Media with these T cell activators was used as negative control (NC) and PBMC from a healthy donor was used as positive control (PC). After incubation, the supernatant was aspirated and the wells were washed twice with deionized water followed by washes with wash buffer A (1×PBS containing 0.05% Tween-20). Wells were incubated for 2 hours at room temperature with detection antibody (1:250 dilution in 1×PBS containing 10% FBS, Mabtech #3420-6-250). Wells were then washed with wash buffer A and incubated with Streptavidin-Horseradish Peroxide (1:100 dilution in 1×PBS containing 10% FBS, BD Bioscience #557630) for 1 hour at room temperature. Streptavidin-Horseradish Peroxide solution was discarded and wells were washed with wash buffer A and wash buffer B (1×PBS). AEC substrate solution (1 drop of AEC chromogen per 1 ml AEC substrate, BD Bioscience #551951) was added to each well and monitored for spot development from 5 min to 20 min maximum. Reactions were stopped using deionized water and the plate was air dried prior to enumeration of spots using an Elispot reader. Data obtained were analyzed using Graphpad Prism version 7.

Results

Patients

A total of 33 patients were treated within a 31 month period. Demographics and baseline clinical characteristics are presented in Table 2. Six, 5, 5, and 17 patients received the lowest, lower, higher, and highest exposures, respectively.

TABLE 2
Demographics and Baseline Disease Characteristics

| Characteristic | IGV-001 (n = 33) | |
|---|---|---|
| Sex, n (%) | | |
| Male | 20 | (60.6) |
| Female | 13 | (39.4) |
| Age, y | | |
| Mean (SD) | 60.2 | (10.5) |
| Median (range) | 63 | (32-77) |
| Extent of gross resection, n (%) | | |
| Total (100%)* | 10 | (30.3) |
| Near total (95%-99%) | 7 | (21.2) |
| Subtotal (>biopsy, <95%) | 16 | (48.5) |
| KPS, n (%) | | |
| 90-100 | 26 | (78.8) |
| 70-80 | 6 | (18.2) |
| 60 | 1 | (3.0) |
| MGMT status at diagnosis, n (%) | | |
| Methylated | 16 | (48.5) |
| Unmethylated | 17 | (51.5) |

KPS = Karnofsky Performance Status;
MGMT = O⁶-methylguanine-DNA methyltransferase
*Complete removal of the enhancing nidus of the tumor. There is never a complete resection of this infiltrating tumor with a non-enhancing periphery that is unresectable.

Safety

IGV-001 was generally well tolerated. As of a date more than 41 months following the first patient treated, there were 5 AEs related to the abdominal incision: one grade 3 hematoma, 3 grade 2 hematomas, and one grade 1 wound complication. There were no documented abdominal wound infections. There were 8 AEs that were possibly related to treatment: 2 grade 3 seizures, one grade 3 DVT, one grade 3 hydrocephalus, one grade 3 elevated alanine aminotransferase (ALT), one grade 3 elevated aspartate aminotransferase (AST), one grade 3 encephalopathy, and one grade 2 DVT.

Eleven of 22 deaths were not attributed to glioblastoma progression. Seven of those 11 deaths occurred within 12 months of treatment with IGV-001 and are detailed in Table 3.

TABLE 3
Deaths Occurring Within 12 Months of IGV-001 Vaccination
and Not Attributable to Glioblastoma Progression

| | | Cause of Death and | OS |
|---|---|---|---|
| Patient | Treatment Group | Relationship to Treatment | (Months) |
| TJ02-31 | 20 chambers/24 hrs | Stercoral colitis and sigmoid | 3.7 |
| | | colon perforation. Unrelated. | |
| TJ07-42 | 10 chambers/48 hrs | Thrombocytopenia. Unrelated. | 9.1 |
| TJ08-52 | 20 chambers/48 hrs | Sacral decubitus ulcer. | 5.5 |
| | | Unrelated. | |
| TJ09-23 | 10 chambers/24 hrs | Stroke. Unrelated. | 8.6 |
| TJ23-46 | 20 chambers/48 hrs | Sepsis. Unrelated. | 7.3 |
| TJ29-27 | 20 chambers/48 hrs | Unknown. Unrelated. | 6.5 |
| TJ31-47 | 20 chambers/48 hrs | Unknown. Unrelated. | 3.8 |

The median (range) follow-up for patients receiving IGV-001 was 13 months (4-40). Radiographic responses included sustained lack of anatomic enhancement after gross total resection, sustained regression from initial post-operative tumor volume, and increases in anatomic tumor volume after subtotal resection followed by sustained regression beginning within ˜6 months of surgery. Two patients also demonstrated spontaneous remission of recurrent tumors (not shown). Tumor recurrences have been typically ipsilateral and considered local (within 2 cm of the original focus). Of the 11 surviving patients, 6 are progression-free according to RANO criteria at last observation.

Clinical Outcomes

The current standard of care (SOC) for suspected glioblastoma begins with maximal safe resection; pathologically confirmed cases then receive concurrent radiotherapy and temozolomide (TMZ), followed by maintenance TMZ.6 This SOC was established by a 2005 study conducted by the European Organisation for Research and Treatment of Cancer under the leadership of Roger Stupp. This trial demonstrated that adding temozolomide (TMZ) to radiation therapy extended OS over radiation alone (14.6 vs 12.1 months). There is a remarkable consistency in OS among SOC arms of large, randomized clinical trials published over 8 years in high-tier, peer-reviewed journals.

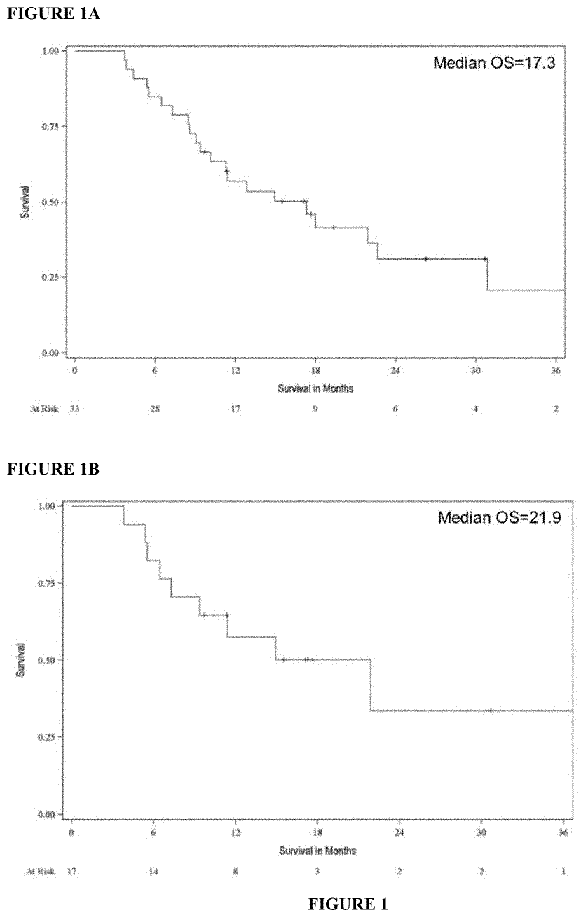

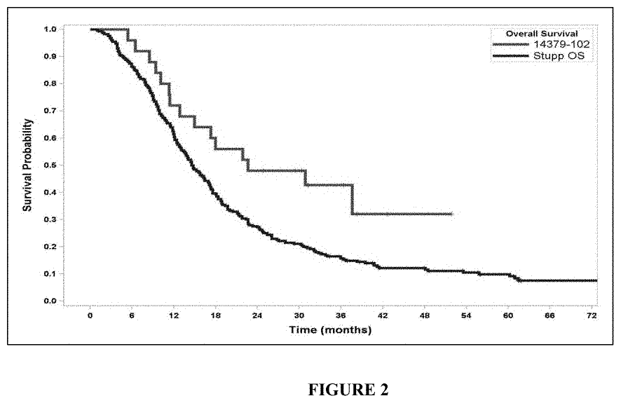

As of a date more than 41 months following the first patient treated, 11 patients treated with IGV-001 were alive and functioning well. Median OS was 17.3 months (FIG. 1A), and OS rate at 24 months was 31%. The OS benefit of IGV-001 was enhanced when analyses were limited to patients receiving the highest exposure of IGV-001 (median, 21.9 months; FIG. 1B).

OS is summarized by exposure group in Table 4. A survival advantage was associated with the highest exposure to IGV-001 (ie, 20 chambers for 48 hours).

TABLE 4
OS Treatment Group (ITT Subjects)

| Treatment Group | Median OS(Months) | 2-Year OS |
|---|---|---|
| 10 Chambers/24 hrs (n = 6) | 20.0 | 25% |
| 10 Chambers/48 hrs (n = 5) | 10.1 | 20% |
| 20 Chambers/24 hrs (n = 5) | 12.8 | 40% |
| 20 Chambers/48 hrs (n = 17) | 21.9 | 34% |
| Total (N = 33) | 17.3 | 31% |

Stupp et al indicated that 95% of all patients receiving SOC for newly-diagnosed glioblastoma experienced tumor progression prior to death. In our study, 14 patients died within the first year; 7 (50%) without disease progression (Table 2). We evaluated the OS of IGV-001-treated patients independent of the mortality likely caused by SOC by excluding deaths not attributable to disease progression. The resultant median OS was 22.1 months, which contrasts favorably with published estimates for SOC.

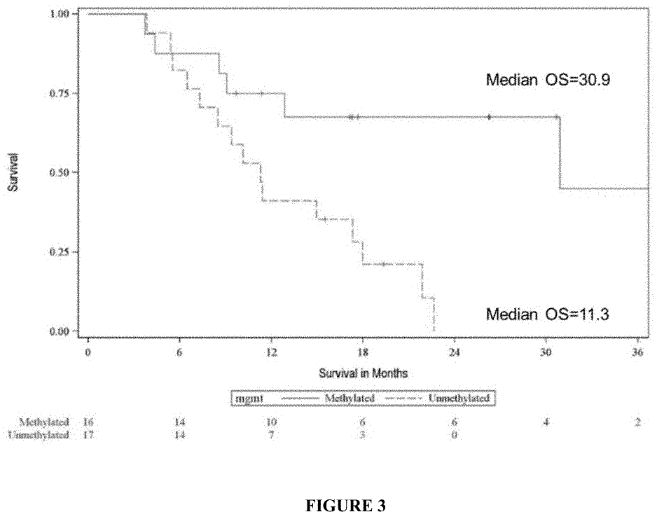

We also evaluated other trial subgroups with clinical characteristics that might favor better OS. Methylation of the 06-methylguanine-DNA methyltransferase (MGMT) promoter silences the ability of a cell to dealkylate the methyl group on 06 guanine and increases the therapeutic efficacy of TMZ compared to patients with an unmethylated MGMT promoter. A companion paper to the Stupp trial examined the effect of MGMT promoter on survival outcomes in patients receiving SOC. Median OS in patients with methylated MGMT promoter was 21.7 months, versus 12.7 months in unmethylated patients. Similarly, we noted a survival advantage in patients with methylated MGMT promotor (median OS, 30.9 vs 11.3 months) treated with IGV-001 followed by SOC (FIG. 3).

As of a date more than 41 months following the first patient treated, 15 patients were progression-free at the primary site, 6 of whom were alive. Median PFS in patients treated with IGV-001 was 10.4 months, and PFS rate at 6 months was 87% (Table 5).

TABLE 5
PFS Treatment Group (ITT Subjects)

| Treatment Group | Median PFS (Months) | 6-Month PFS |
|---|---|---|
| 10 Chambers/24 hrs (n = 6) | 11.6 | 67% |
| 10 Chambers/48 hrs (n = 5) | 9.5 | 80% |
| 20 Chambers/24 hrs (n = 5) | 19.0 | 100% |
| 20 Chambers/48 hrs (n = 17) | 10.4 | 93% |
| Total (N = 33) | 10.4 | 87% |

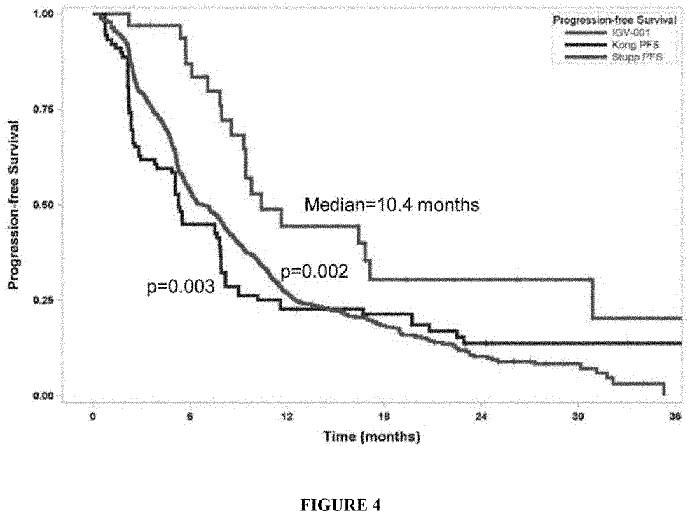

Because PFS data mature more quickly than do OS data, we compared our PFS results to the SOC arms of 2 large, randomized clinical trials published in high-tier, peer-reviewed journals. We noted significant improvements in PFS with IGV-001 versus SOC (FIG. 4).

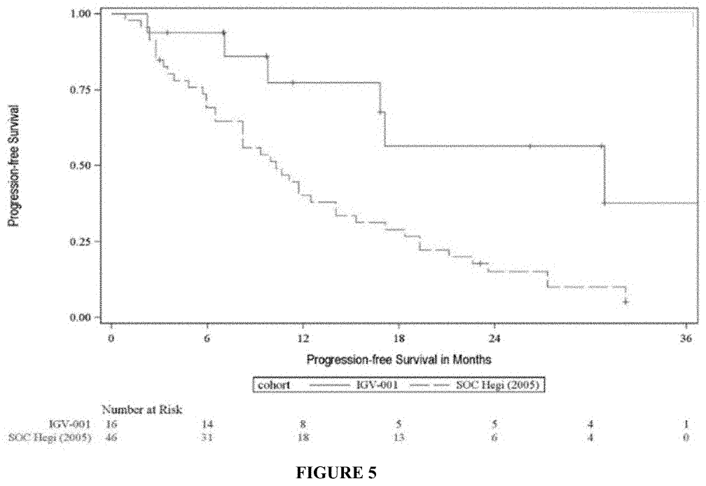

Hegi, Gilbert, and Stupp reported median PFS of 10.3, 10.5, and 10.7 months, respectively, in patients with methylated MGMT promoter receiving SOC alone. The median PFS among patients with methylated MGMT promoter receiving IGV-001 was 30.9 months (Table 6), which compares favorably to SOC (p=0.004; FIG. 5).

TABLE 6
PFS by MGMT Promoter Methylation Status (ITT Subjects)

| Methylation Status | Median PFS (months) | 6-Month PFS |
|---|---|---|
| Methylated | 30.9 | 94% |
| Unmethylated | 9.3 | 80% |

Initial Review of Predictive Biomarkers

In preliminary analyses, good T cell function (ie, median or greater number of T cells expressing IFN-γ in response to nonspecific stimulation) prior to treatment was associated with greater OS than was poor T cell function (ie, less than median; Table 7), suggesting immune system involvement in the mechanism of action of IGV-001.

TABLE 7
OS in Patients with Good and Poor T Cell
Function* Prior to Treatment with IGV-001

| T Cell Function | Median OS (Months) | 2-Year OS |
|---|---|---|
| Good (n = 15) | 21.9 | 38% |
| Poor (n = 14) | 10.1 | 27% |

*T cell function was based median or greater (good) or less than median (poor) number of T cells expressing IFN-γ in response to nonspecific stimulation. Data were not available for 4 patients.

### CONCLUSIONS

The results of this Phase 1b clinical trial of IGV-001 in patients with newly diagnosed glioblastoma are compelling. The combination vaccine was implanted in 33 patients and was generally well tolerated. Seven of 14 deaths in the first year occurred in the absence of disease progression; all were unrelated to treatment. Median OS and PFS compared favorably with SOC reported in large clinical trials. The highest exposure to IGV-001, methylation of the MGMT promoter, and good T cell function prior to treatment were associated with longer survival.

### REFERENCES

1. Andrews D W, Resnicoff M, Flanders A E, et al. Results of a pilot study involving the use of an antisense oligodeoxynucleotide directed against the insulin-like growth factor type I receptor in malignant astrocytomas. J Clin Oncol. 2001; 19(8):2189-2200.
2. Wen P Y, Chang S M, Van den Bent M J, Vogelbaum M A, Macdonald D R, Lee E Q. Response Assessment in Neuro-Oncology Clinical Trials. J Clin Oncol. 2017; 35(21):2439-2449.
3. Okada H, Weller M, Huang R, et al. Immunotherapy response assessment in neuro-oncology: a report of the RANO working group. Lancet Oncol. 2015; 16(15):e534-e542.
4. Harshyne L A, Hooper K M, Andrews E G, et al. Glioblastoma exosomes and IGF-1R/AS-ODN are immunogenic stimuli in a translational research immunotherapy paradigm. Cancer Immunol Immunother. 2015; 64(3):299-309.
5. Morin-Brureau M, Hooper K M, Prosniak M, et al. Enhancement of glioma-specific immunity in mice by “NOBEL”, an insulin-like growth factor 1 receptor antisense oligodeoxynucleotide. Cancer Immunol Immunother. 2015; 64(4):447-457.
6. National Comprehensive Cancer Network. NCCN Clinical Practice Guidelines in Oncology: Central Nervous System Cancers Version 1.2018; https://www.nccn.org/professionals/physician_gls/pdf/cns.pdf.
7. Stupp R, Mason W P, van den Bent M J, et al. Radiotherapy plus concomitant and adjuvant temozolomide for glioblastoma. N Engl J Med. 2005; 352(10):987-996.
8. Chinot O L, Wick W, Mason W. et al. Bevacizumab plus radiotherapy-temozolomidc for newly diagnosed glioblastoma. N Engl J Med. 2014; 370(8):709-722.
9. Gilbert M R. Dignam J J, Armstrong T S, et al. A randomized trial of bevacizumab for newly diagnosed glioblastoma. N Engl J Med. 2014; 370(8):699-708.
10. Kong D S, Nam D H, Kang S H, et al. Phase III randomized trial of autologous cytokine-induced killer cell immunotherapy for newly diagnosed glioblastoma in Korea. Oncotarget. 2017; 8(4):7003-7013.
11. Stupp R, Hegi M E, Mason W P, et al. Effects of radiotherapy with concomitant and adjuvant temozolomide versus radiotherapy alone on survival in glioblastoma in a randomised phase III study: 5-year analysis of the EORTC-NCIC trial. Lancet Oncol. 2009; 10(5):459-466.
12. Hegi M E, Diserens A C, Gorlia T, et al. MGMT gene silencing and benefit from temozolomide in glioblastoma. N Engl J Med. 2005; 352(10):997-1003.
13. Gilbert M R, Wang M, Aldape K D, et al. Dose-dense temozolomide for newly diagnosed glioblastoma: a randomized phase III clinical trial. J Clin Oncol. 2013; 31(32):4085-4091.
14. Stupp R, Hegi M E, Gorlia T, et al. Cilengitide combined with standard treatment for patients with newly diagnosed glioblastoma with methylated MGMT promoter (CENTRIC EORTC 26071-22072 study): a multicentre, randomised, open-label, phase 3 trial. Lancet Oncol. 2014; 15(10):1100-1108.

### INCORPORATION BY REFERENCE

All patents and publications referenced herein are hereby incorporated by reference in their entireties.

1. A method comprising
identifying a subject having cancer and having an increased likelihood of responding to an IGF-1R AS ODN or treatment with the IGF-1R AS ODN and
administering to the subject the IGF-1R AS ODN;
wherein the increased likelihood of responding to an IGF-1R AS ODN is evaluated by at least one selected from the group consisting of:
a) determining MGMT methylation in the subject; and
b) determining T-cell function in the subject.

2. The method of claim 1, wherein the increased likelihood is established by at least one selected from the group consisting of:
a) identifying MGMT methylation in the subject; and
b) determining good T cell function in the subject.

3.-4. (canceled)

5. A method of predicting the prognosis of a subject having cancer in response to an IGF-1R AS ODN; the method comprising at least one selected from the group consisting of:
a) determining MGMT methylation in the subject; and
b) determining T-cell function in the subject.

6. The method of claim 5, wherein
a) methylated MGMT in the subject is indicative of a favorable prognosis;
b) good T cell function in the subject is indicative of a favorable prognosis;
c) methylated MGMT and good T cell function in the subject is indicative of a favorable prognosis;
d) unmethylated MGMT in the subject is indicative of an unfavorable prognosis;
e) poor T cell function in the subject is indicative of an unfavorable prognosis; or
f) unmethylated MGMT and poor T cell function in the subject is indicative of an unfavorable prognosis.

7.-11. (canceled)

12. The method of claim 1, wherein the T cell function is determined by evaluating the number of T cells expressing IFN-γ in response to stimulation.

13. (canceled)

14. The method of claim 1, wherein the IGF-1R AS ODN is administered to subject before temozolamide is administered to the subject.

15. (canceled)

16. The method of claim 1, wherein the IGF-1R AS ODN is administered to the subject as an autologous cancer cell vaccine.

17. The method of claim 1, wherein the IGF-1R AS ODN is administered to the subject as a fully formulated biodiffusion chamber.

18. The method of claim 17, wherein the biodiffusion chamber is prepared by:
(a) encapsulating tumor cells obtained from the subject into the biodiffusion chamber in the presence of an IGF-1R AS ODN,
wherein the ratio of tumor cells to IGF-1R AS ODN in the chamber is in a range from about 3.75×10⁵: 1 μg to about 6.25×10⁵: 1 μg;
wherein the tumor cells are obtained from the subject, and
(b) irradiating the biodiffusion chamber.

19. The method of claim 18, wherein the tumor cells are enriched for nestin expression before they are placed into the biodiffusion chamber.

20. The method of claim 18, wherein the tumor cells in the chamber are enriched for adherent cells compared to the tumor cells obtained from the subject.

21. (canceled)

22. The method of claim 18, wherein the cells are treated with IGF-1R AS ODN before encapsulation into the chamber.

23.-26. (canceled)

27. The method of claim 1, wherein the IGF-1R AS ODN has the sequence of SEQ ID NO:1.

28.-30. (canceled)

31. The method of claim 18, wherein the method comprises implanting two or more biodiffusion chambers into the subject.

32.-34. (canceled)

35. The method of claim 1, wherein the cancer is a brain cancer.

36. (canceled)

37. The method of claim 1, wherein the subject is a human.

38. The method of claim 5, wherein the T cell function is determined by evaluating the number of expressing IFN-γ in response to stimulation.

39. The method of claim 5, wherein the IGF-1R AS ODN is administered to subject before temozolamide is administered to the subject.

40. The method of claim 5, wherein the IGF-1R AS ODN is administered to the subject as an autologous cancer cell vaccine.

41. The method of claim 5, wherein the IGF-1R AS ODN is administered to the subject as a fully formulated biodiffusion chamber.

42. The method of claim 5, wherein the IGF-1R AS ODN has the sequence of SEQ ID NO:1.

43. The method of claim 5, wherein the cancer is a brain cancer.

44. The method of claim 5, wherein the subject is a human.
